(19) United States
(12) Patent Application Publication
(10) Pub. No.: US 2022/0280486 A1
(43) Pub. Date: Sep. 8, 2022

(54) COMBINED CANCER THERAPY INVOLVING CHEMICAL ACTIVATION OF INTEGRINS AND TARGETED CELL IMMUNOTHERAPY

(71) Applicant: The Regents of the University of California, Oakland, CA (US)

(72) Inventors: Ronald D. VALE, Tiburon, CA (US); Meghan A. MORRISSEY, San Francisco, CA (US)

(21) Appl. No.: 17/632,183

(22) PCT Filed: Aug. 14, 2020
(86) PCT No.: PCT/US20/46467

§ 371 Date: Feb. 1, 2022

Related U.S. Application Data
(60) Provisional application No. 62/887,978, filed on Aug. 16, 2019.

(51) Int. Cl. A61K 31/427; A61K 39/395; A61K 35/15

(52) U.S. Cl. CPC A61K 31/427; A61K 39/3955; A61K 35/15

(57) ABSTRACT

The present disclosure relates generally to novel approaches to activate integrin signaling in order to overcome CD47 checkpoint inhibition and to promote macrophage phagocytic signaling pathway. The disclosure also provides methods and compositions for treatment of cancer, including solid tumor and hematologic malignancy, by promoting macrophage-mediated engulfment of cancer cells. Use of integrin activation in combination with adoptive transfer of engineered macrophages to increase engulfment of cancer cells is also provided.

### CROSS-REFERENCE TO RELATED APPLICATIONS

This application claims the benefit of priority to U.S. Provisional Patent Application Ser. No. 62/887,978, filed on Aug. 16, 2019. The disclosure of the above-referenced application is herein expressly incorporated by reference it its entirety, including any drawings.

### FIELD

The present disclosure relates generally to novel approaches to activate integrin signaling in order to overcome CD47 checkpoint inhibition. The disclosure also provides methods for treatment of cancer, including solid tumor and hematologic malignancy, by promoting macrophage-mediated engulfment of cancer cells. Use of integrin activation in combination with a targeted cancer therapy such as adoptive transfer of engineered macrophages to increase engulfment of cancer cells is also disclosed.

### BACKGROUND

The success of cancer immunotherapy has generated increasing interest in identifying new immunotherapeutic targets. To date, the majority of therapies have focused on stimulating the adaptive immune system to attack cancer, including agents targeting CTLA-4 and the PD-1/PD-L1 axis. However, recent advances in cancer immunotherapy offer tremendous opportunities to use macrophages and other myeloid immune cells as promising effectors of cancer immunotherapy. The CD47/signal regulatory protein alpha (SIRPα) axis is an important regulator of myeloid cell activation and serves a broader role as a myeloid-specific immune checkpoint. CD47 is highly expressed on many different types of cancer, and it transduces inhibitory signals through SIRPα on macrophages and other myeloid cells. In a diverse range of preclinical models, therapies that block the CD47/SIRPα axis stimulate phagocytosis of cancer cells in vitro and anti-cancer immune responses in vivo.

A number of therapeutics that target the CD47/SIRPα axis are under preclinical and clinical investigation for both solid and hematologic malignancies using anti-CD47 antibodies and recombinant SIRPα proteins. These include anti-CD47 antibodies, engineered receptor decoys, anti-SIRPα antibodies and bispecific agents. However, while monoclonal antibodies are now clinically important in the treatment of cancer, particularly leukemias, there remains considerable need for improvement in therapeutic methods.

### SUMMARY

The present disclosure relates generally to novel combination approaches for cancer therapy. The combination includes two elements, the first is an integrin agonist which activates one or more α and/or β integrin subunits. The second element is a targeted cancer therapy such as, an antibody therapy, a chimeric antigen receptor T-cell (CAR-T) therapy, or a chimeric antigen receptor for phagocytosis (CAR-P) therapy, which targets at least one cancer-associated antigen and/or cancer-specific antigen. In some particular embodiments, provided herein are methods for activating integrin signaling in order to overcome CD47 checkpoint inhibition. In some embodiments, the disclosed methods further promote macrophage phagocytic signaling pathway. Some embodiments of the disclosure also provide methods for treatment of cancer, including solid tumor and hematologic malignancy, in an individual by promoting macrophage-mediated engulfment of cancer cells. In certain embodiments, the use of integrin activation in combination with one or more targeted cancer therapy, such as antibody therapies, chimeric antigen receptor T-cell (CAR-T) therapies, chimeric antigen receptor for phagocytosis (CAR-P) therapies, adoptive transfer macrophages is provided.

In one aspect, provided herein are various methods for treatment of a cancer in an individual in need thereof. The methods include administering to the individual (a) a first therapy comprising a therapeutically effective amount of an integrin agonist; and (b) a second therapy comprising a cancer therapy that targets at least one cancer-associated antigen and/or cancer-specific antigen.

Non-limiting exemplary embodiments of the methods according to the present disclosure include one or more of the following features. In some embodiments, the agonist activates one or more integrins selected from the group consisting of α integrins, β integrins, and combinations of any thereof. In some embodiments, the integrin agonist activates αVβ3, αLβ2, αMβ2, αXβ2, αDβ2, α4β1, α4β7, αEβ7, or a combination of any thereof.

In some embodiments, the integrin agonist includes a manganese treatment, a high affinity integrin ligand, a small molecule agonist, or a combination of any thereof. In some embodiments, the small molecule agonist of integrin includes leukadherin-1 (LA1), ADH-503, or a combination thereof. In some embodiments, the high affinity integrin ligand includes ICAM-1, ICAM-2, ICAM-3, VCAM-1, MAdCAM-1, E-cadherin, JAM-1, JAM-2, JAM-3, or a combination of any thereof.

In some embodiments, the cancer is a solid tumor or a hematologic malignancy. In some embodiments, the cancer is selected from the group consisting of leukemia, pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, lymphoma, and glioblastoma.

In some embodiments, the targeted cancer therapy includes an antibody therapy, a chimeric antigen receptor T-cell (CAR-T) therapy, a chimeric antigen receptor for phagocytosis (CAR-P) therapy, a myeloid-targeting therapy, or a combination thereof, that targets at least one cancer-associated antigen and/or cancer-specific antigen. In some embodiments, the targeted cancer therapy comprises one or more phagocytic cells expressing a CAR that comprises an intracellular signaling domain of the engulfment receptor. In some embodiments, the intracellular signaling domain of the engulfment receptor comprises at least 1, at least 2, at least 3, at least 4, or at least 5 ITAM motifs. In some embodiments, the intracellular signaling domain from the engulfment receptor is capable of mediating endogenous phagocytic signaling pathway. In some embodiments, the engulfment receptor is selected from the group consisting of Megf10, FcRγ, Bai1, MerTK, TIM4, Stabilin-1, Stabilin-2, RAGE, CD300f, Integrin subunit αv, Integrin subunit β5, CD36, LRP1, SCARF1, C1Qa, and Ax1. In some embodiments, the one or more phagocytic cells is selected from the group consisting of macrophages, dendritic cells, mast cells, monocytes, neutrophils, microglia, and astrocytes. In some embodiments, at least one of the one or more phagocytic cells is a bone marrow derived macrophage (BMDM) or a bone marrow derived dendritic cell (BMDC).

In some embodiments, the targeted cancer therapy is an antibody therapy including an anti-CD47 antibody, an anti-SIRPα antibody, or a combination thereof.

In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

In another aspect, provided herein are kits for the treatment of a cancer in a subject in need thereof, the kit including one or more integrin agonists and instructions for use of the one or more integrin agonists in combination with a cancer therapy that targets a cancer-associated antigen and/or a cancer-specific antigen.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1E schematically summarize the results of experiments performed to demonstrate that CD47-SIRPA suppresses IgG and PS dependent engulfment. FIG. 1A: Schematic shows the supported lipid bilayer system used in this study. Anti-biotin IgG is bound to biotinylated lipids. IgG is recognized by Fc Receptor in the macrophage. The extracellular domain of CD47-His10 is bound to Ni-NTA-conjugated lipids and recognized by SIRPA expressed in the macrophage. FIG. 1B: Silica beads are coated with a supported lipid bilayer and incubated with the indicated concentration of IgG and either CD47 (red) or an inactive mutant CD47 (F37D, T115K; green). The functionalized beads were added to RAW264.7 macrophages and fixed after 30 min. The average number of beads per macrophage was assessed by confocal microscopy and normalized to the maximum average bead eating observed in that replicate. Each dot represents an independent replicate (n≥100 cells analyzed per experiment), and the data are compared using a two-way ANOVA. For visualization, the data were fit using the “[Agonist] vs response—variable slope” model in Graphpad Prism. FIG. 1C: Still images depict the assay described in FIG. 1B. The supported lipid bilayers contain the fluorescently-labeled lipid atto390-DOPE (green) and the macrophages membranes are labeled with CellMask (magenta). Internalized beads are indicated with a yellow dot. FIG. 1D: Graph depict the fraction of cells engulfing the indicated number of beads (pooled data from the three independent replicates included in FIG. 1B). Macrophages encountering CD47-conjugated beads (right) were less likely to engulf, and those that did engulfed fewer beads. CD47^{F37D,T115K}, a mutant that cannot bind SIRPA, was used as a control. FIG. 1E: Macrophages were incubated with beads coated in a supported lipid bilayer containing 10% phosphatidylserine and either CD47 or the inactive CD47^{F37D,T115K} Dots and error bars denote the mean and standard error of independent replicates. *** indicates p<0.0005 by a Kruskal-Wallis test on the pooled data (FIGS. 1B and 1E). Scale bar denotes 5 μm in this and all subsequent figures.

FIGS. 2A-2E depict a reconstitution system for studying CD47-SIRPA signaling.

FIG. 2A: SDS page gel shows the N-terminal extracellular domain of murine CD47 purified from insect cells using a C-terminal His₁₀. FIG. 2B: Beads coated in supported lipid bilayers were incubated with the indicated concentration of anti-biotin IgG. The fluorescent intensity of Alexa Fluor 647-IgG on the bead was measured to ensure that the binding of IgG increased with higher coupling concentrations. FIG. 2C: The estimated surface density of CD47 on red blood cells (Gardner et al., 1991; Mouro-Chanteloup et al., 2003), T cells (Subramanian et al., 2006), cancer cells (Dheilly et al., 2017; Jaiswal et al., 2009; Michaels et al., 2017) and the beads used in this study. FIG. 2D: IgG surface density was held constant while CD47 density was titrated. The 1 nM CD47 coupling concentration was selected for use throughout this study. FIG. 2E: Histograms depict the fraction of macrophages engulfing the indicated number of phosphatidylserine beads. RAW264.7 engulfment was measured after 30 min and J774A.1 was measured after 90 min.

FIGS. 3A-3G schematically summarize the results of experiments performed to demonstrate that forcing SIRPA into the macrophage-target synapse suppresses engulfment.

FIG. 3A: SIRPA-GFP (top; green in merge) is depleted from the base of the phagocytic cup (arrow) when a macrophage engulfs an IgG-coated beads (left; supported lipid bilayer, magenta), but not when CD47 is present (IgG+CD47, right). Graph depicts the ratio of SIRPA-GFP at the phagocytic cup/cell cortex for individual phagocytic cups. FIG. 3B: A schematic shows the chimeric SIRPA with a small extracellular domain (FRB^{ext}-SIRPA). FRB^{ext}-SIRPA-GFP fluorescence is shown at cell-bead contacts and graphed on the right (compare to SIRPA-GFP in FIG. 3D). FIG. 3C: Schematic shows chimeric SIRPA construct used to target SIRPA to the cell synapse. FIG. 3D: SIRPA-GFP (left) and the chimeric receptor FcRI^{ext}-SIRPA^{int}-GFP (center) are shown at the phagocytic synapse (arrow). The ratio of fluorescence at the bead contact site/cell cortex is graphed on the right. FIG. 3E: A graph depicts the average number of internalized IgG beads per macrophage expressing the constructs schematized in FIG. 3C, normalized to macrophages expressing only a membrane-tethered GFP (GFP-CAAX). FIG. 3F: Schematic (left) shows a system for inducible recruitment of the SIRPA intracellular domain to the phagocytic cup. Recruiting SIRPA to the phagocytic cup suppresses engulfment compared to soluble SIRPA or compared to wild-type macrophages treated with rapamycin (normalized to uninfected macrophages). FIG. 3G: The graph shows the number of beads engulfed by uninfected, SIRPA-GFP or FRB^{ext}-SIRPA expressing macrophages normalized to uninfected cells. In FIGS. 3A, 3B, and 3D, dots represent individual cups, lines show mean±SD and data is pooled from three independent experiments. In FIGS. 3E, 3F, and 3G, dots show the average from an independent replicate with the error bars denoting SEM for that replicate. The complete pooled data showing the number of beads eaten per macrophage is shown in FIGS. 4A-4F. The non-activating CD47^{F37D,T115K} was used as a control on bilayers lacking CD47 in FIG. 3A. ** denotes p<0.0005, ** denotes p<0.005 and * denotes p<0.05 as determined by a Student's T test (FIGS. 3A, 3B, and 3D) or a Kruskal-Wallis test on the pooled data from all three replicates (FIGS. 3E, 3F, and 3G).

FIGS. 4A-4F summarize the results of experiments performed to demonstrate that forcing SIRPA into the macrophage-target synapse suppresses engulfment. FIG. 4A: Schematic depicts TIRF imaging. FIG. 4B: TIRF microscopy of J774A.1 macrophages encountering a 10% phosphatidylserine bilayer reveals that SIRPA-GFP is depleted at the center off the cell-bilayer synapse (top; yellow arrow compared to cyan arrow). Macrophages did not form this zone of depletion when encountering a bilayer containing both phosphatidylserine and CD47 (bottom). The ratio of SIRPA-GFP fluorescent intensity at the cell center/cell edge is quantified on the right. Each dot represents an individual cell and data is pooled from 3 independent experiments. Lines denote the mean±SD. *** denotes p<0.0005 by Student's T test. FIG. 4C: SIRPA-GFP and the chimeric receptors FcRI^{ext}-SIRPA^{int}-GFP and FRB^{ext}-SIRPA are expressed at similar levels. Fluorescent intensity was normalized to the average intensity of SIRPA-GFP in that experiment. Each dot represents an individual cell and data is pooled from 3 independent experiments. Lines denote the mean±SD. FIGS. 4D, 4E, 4F: Histograms depict the fraction of macrophages engulfing the indicated number of IgG-bound beads. The average number of beads per cell is shown ±SEM. This data corresponds to 1E (FIG. 4D), 1F (FIG. 4E) and 1G (FIG. 4F). For all panels, data is pooled from three data is pooled from 3 independent experiments. Lines denote the mean±SD.

FIGS. 5A-5D schematically summarize the results of experiments performed to demonstrate that CD47 prevents integrin activation. FIG. 5A: Still images from a TIRF microscopy timelapse show that macrophages form IgG (black) microclusters as they spread across bilayers containing IgG and an inactive CD47^{F37D,T115K} which cannot bind to SIRPA bilayer (top). Adding CD47 to the bilayer inhibits cell spreading (bottom; graphed on right, average area of contact from n≥11 cells ±SEM, pooled from three separate experiments). FIG. 5B: TIRF images show the cell membrane (mCherry-CAAX; white) of macrophages engaging with an IgG and inactive CD47^{F37D,T115K} (left) or IgG and CD47 (right) bilayer. Graphs depict the average number of cells seen contacting the bilayer after 10 min (center) and the average area of cell contact (right). Each dot represents an individual field of view (center) or cell (right) pooled from three independent experiments. FIG. 5C: Blocking integrin activation using a function-blocking antibody (2E6) targeting the β2 integrin subunit decreased the efficiency of engulfment. Graph shows the number of beads engulfed normalized to the maximum observed eating in that replicate. Each data point represents an independent experiment and the error bars denote the SEM for that replicate. FIG. 5D: Immunofluorescence images show phosphopaxillin (top; green in merge) and F-actin (center; magenta in merge; visualized with phalloidin) at the phagocytic cup of a bead containing IgG and inactive CD47^{F37D,T115K} (left) or an IgG- and CD47-coated bead (right). Graphs show the ratio of phosphopaxilin (center) or actin (right) intensity at the phagocytic cup/cell cortex. Each dot represents an individual phagocytic cup; lines denote the mean±95% confidence intervals. *** denotes p<0.0005, ** denotes p<0.005, and * denotes p<0.05 as determined by Student's T test (FIGS. 5A, 5B, and 5C) or Ordinary one-way ANOVA with Tukey's Multiple Comparison test (FIG. 5D).

FIGS. 6A-6C summarize the results of experiments performed to show that CD47 does not affect FcR activation and Syk recruitment. FIG. 6A: TIRF microscopy shows that macrophages are able to form IgG microclusters (left; cyan in merged image) that recruit Syk (middle; magenta in merged image) if CD47 is absent (top) or present (bottom). Inset shows the boxed region of the image above. The linescan shows the fluorescent intensity of Alexa Fluor 647-IgG and Syk-mCherry at the indicated position (white arrow). Intensity was normalized so that 1 is the highest observed intensity and 0 is background. The fraction of cells able to form IgG clusters and recruit Syk is displayed on the far right. Each dot represents the percent from an independent experiment (n≥20 per replicate) and the lines denote mean±SD. FIG. 6B: TIRF microscopy shows that, in the presence of CD47, SIRPA (green) does not co-localize with IgG clusters (cyan; arrowheads). Inset shows the boxed region in the above image. The linescan shows the fluorescent intensity of Alexa Fluor 647-IgG and SIRPA-GFP at the position indicated by a white arrow. FIG. 6C: Macrophages were incubated with a Fab generated from the β2 function-blocking antibody (2E6, red) or from an isotype control (green). The pooled data from three independent replicates is graphed with error bars denoting SEM. ** indicates p<0.005 by Kruskal-Wallis test.

FIGS. 7A-7I pictorially summarize the results of experiments performed to demonstrate that bypassing inside out activation of integrin eliminates the effect of CD47. FIG. 7A: The schematic shows a simplified signaling diagram. If CD47 and SIRPA act upstream of integrin, then providing an alternate means of integrin activation (Mn2⁺ or ICAM) should eliminate the effect of CD47. FIG. 7B: Macrophages were treated with 1 mM Mn2+ and fed beads with IgG and either CD47 (red) or the non-signaling CD47F37D, TI15K (green). Bars denote the average number of beads eaten from the pooled data of three independent replicates ±SEM. FIG. 7C: Beads were incubated with the indicated concentration of IgG and added to macrophages. Treatment with Mn2+ did not dramatically enhance engulfment (black, compared to grey). Dots represent the average number of beads eaten ±SEM in one data set representative of three experiments. FIG. 7D: Immunofluorescence shows that adding ICAM (10 nM coupling concentration) to IgG+CD47 beads rescues phosphopaxillin (top; green in merge, bottom) at the phagocytic cup. Compare to data displayed in FIG. 5D (p<0.0005 for phosphopaxilin with ICAM and CD47 compared to CD47 alone). FIG. 7E: Beads were functionalized with IgG and either CD47 (red) or the non-signaling CD47F37D, T 115K (green). Adding ICAM to the beads abrogated the effect of CD47 (center) but did not stimulate engulfment without IgG (right). FIG. 7F: ICAM also rescued actin accumulation at the phagocytic cup as measured by the ratio of phalloidin fluorescence at the cup to the cell cortex. FIG. 7G: Bone marrow-derived macrophages expressing a membrane tethered GFP (GFP-CAAX) were incubated with L1210 murine leukemia cells expressing H2B-mCherry. Treating with 100 μM manganese allowed for engulfment of whole cancer cells. These images correspond to frames taken from a movie (Movie S3) showing a macrophage (green) engulfs a cancer cell (magenta). The macrophage was a mouse bone marrow derived macrophage expressing GFP-caax labeling the cell membrane. The cancer cells was a mouse leukemia cell line L1210 expressing H2B-mCherry which labeled the nucleus of the cancer cell. It was found that the cancer cell was engulfed and degraded, as can be observed by the release of mCherry from the nucleus. FIG. 7H: The percent of macrophages engulfing a cancer cell during an 8-hour timelapse is graphed. Each dot represents an independent replicate, with lines denoting mean±SEM. FIG. 7I: Model figure shows that in the absence of CD47 (left), SIRPA is segregated away from the phagocytic synapse and Fc Receptor binding triggers inside out activation of integrin. When CD47 is present (right), SIRPA localizes to the synapse and inhibits integrin activation. *** denotes p<0.0005, ** denotes p<0.005 and n.s. denotes p>0.05 as determined by a Kruskal-Wallis test (FIGS. 7B and 7E), Ordinary One-way ANOVA (FIGS. 7D and 7F) or Fisher Exact (FIG. 7H) on the pooled data from all three replicates.

FIG. 8 schematically summarizes the results of experiments performed to demonstrate that Manganese does not affect L1210 viability. In these experiments, L1210 cells were serum starved for 2 hours, then treated with 100 μM manganese for 6 hours as in FIG. 3H. The percent of cells that bound high levels of annexin, indicating phosphatidylserine exposure and the initiation of apoptosis, was measured by flow cytometry.

FIGS. 9A-9J summarize the results of experiments performed to demonstrate that bypassing inside out activation of integrin eliminates the effect of CD47. FIG. 9A: The schematic shows a simplified signaling diagram. If CD47 and SIRPA act upstream of integrin, then providing an alternate means of integrin activation (Mn2+ or ICAM) should eliminate the effect of CD47. FIG. 9B: Macrophages were treated with 1 mM Mn2+ and fed beads with IgG and either CD47 (red) or the non-signaling CD47F37D, T115K (green). Bars denote the average number of beads eaten from the pooled data of three independent replicates ±SEM. FIG. 9C: Beads were incubated with the indicated concentration of IgG and added to macrophages. Treatment with Mn2+ did not dramatically enhance engulfment (black, compared to grey). Dots represent the average number of beads eaten ±SEM in one data set representative of three experiments. FIG. 9D: Immunofluorescence shows that adding ICAM (10 nM coupling concentration) to IgG+CD47 beads rescues phosphopaxillin (top; green in merge, bottom) and actin (middle; magenta in merge) at the phagocytic cup. The quantification of this data is graphed in FIG. 5D alongside the appropriate controls. FIG. 9E: Beads were functionalized with IgG and either CD47 (red) or the non-signaling CD47^{F37D,T115K} (green). Adding ICAM to the beads abrogated the effect of CD47 (center) but did not stimulate engulfment without IgG (right). FIG. 9F: Complement-opsonized CD47+ mouse red blood cells (RBCs) were fed to control (grey) or 1 mM Mn2* treated (black) macrophages. Unopsonized IgM treated RBCs were used as a negative control. Red blood cell internalization is graphed on the left. An anti-iC3b antibody was added after fixation, but before cell permeabilization to distinguish between engulfed (green) and non-engulfed (green and magenta) RBCs. On the right, representative images show increased adhesion and engulfment of RBCs (all RBCs, green; non-engulfed RBCs, magenta) in macrophages (nuclei stained with DAPI, cyan) treated with Mn2⁺. The inset (red box) highlights one example macrophage and is shown at higher magnification on the right. The macrophage cortex was determined through CellMask staining and is outlined in red. FIG. 9G: Schematic shows the DNA-based adhesion system. Macrophages express a synthetic adhesion receptor containing an intracellular GFP, and an extracellular SNAP tag, which is conjugated to benzylguanine DNA. Graph depicts the mean number of bead contacts per cell, using beads functionalized only with neutravidin or with neutravidin and biotinylated ligand DNA (no IgG). Arrows point to cell membrane clinging to the adherent beads. FIG. 9H: Beads were ligated to IgG, either CD47 (red) or the non-signaling CD47^{F37D,T115K} (green), and biotinylated DNA to control adhesion. All cells express the adhesion receptor, which is conjugated to benzylguanine-DNA. Graph depicts the average number of beads engulfed per cell. FIG. 9I: Bone marrow-derived macrophages expressing a membrane tethered GFP (green) were incubated with L1210 murine leukemia cells expressing H2B-mCherry (magenta). Treating with 100 μM manganese allowed for engulfment of whole cancer cells. These images correspond to frames from Movie S3. The percent of macrophages engulfing a cancer cell during an 8-hour timelapse is graphed on the right. Each dot represents an independent replicate, with red lines denoting mean±SEM. FIG. 9J: Model figure shows that in the absence of CD47 (left), SIRPA is segregated away from the phagocytic synapse and Fe Receptor binding triggers inside out activation of integrin. When CD47 is present (right), SIRPA localizes to the synapse and inhibits integrin activation. In 9F, 9G and 9I, each dot represents an independent replicate, with red lines denoting mean±SEM. *** denotes p<0.0005, ** denotes p<0.005 and n.s. denotes p>0.05 as determined by a Kruskal-Wallis test (9B, 9E, 9H), Student's T test (9F, 9G) or an Ordinary one way ANOVA with Holm-Sidak multiple comparison test (9I).

FIGS. 10A-10B schematically summarize the results of experiments performed to illustrate that blockade of β2 or αM integrins disrupts engulfment. FIG. 10A: Macrophages were incubated with a Fab generated from the β2 function-blocking antibody (2E6, red) or from an isotype control (green). Three independent replicates are graphed with error bars denoting SEM. *** indicates p<0.0005 by Student's T-test. FIG. 10B: Macrophages were incubated with a function blocking antibody targeting the indicated integrin subunit, or the relevant isotype control. To remove any potential non-specific integrin ligands, this assay was performed in protein free HEPES-based imaging buffer. The average number of beads eaten per cell was counted and divided by the average beads per cell in the isotype control. Lines indicate the mean±SEM. *** indicates p<0.0005 and ** indicates p<0.005 by Student's T-test comparing the blocking antibody to the isotype control.

FIGS. 11A-11C pictorially summarize the results of experiments performed to illustrate that manganese drives engulfment of viable cancer cells. FIG. 11A: L1210 cells were serum starved for 2 hours, then treated with 100 μM manganese for 6 hrs as in FIGS. 9A-9J. The percent of cells that bound high levels of annexin, indicating phosphatidylserine exposure and the initiation of apoptosis, was measured by flow cytometry. FIG. 11B: L1210 cancer cells were dyed with CFSE and incubated with primary bone marrow derived macrophages for 4 hours at a 2:1 cancer cell:macrophage ratio. Cells were then fixed and stained for F4/80 to label the macrophages and DAPI to label nuclei. Cells that were CFSE and F4/80 double positive, and contained 2 nuclei were scored as an engulfment event. Representative images are shown in FIG. 11C. The red box indicates an engulfment event and is shown at higher magnification on the right. The macrophage cortex is outlined in yellow.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### DETAILED DESCRIPTION OF THE DISCLOSURE

The present disclosure relates generally to, inter alia, therapeutic methods for treatment of cancer, and particularly relates to novel combination approaches for cancer therapy. The combination includes two elements, the first is an integrin agonist which activates one or more α and/or β integrin subunits. The second element is a targeted cancer therapy such as, an antibody therapy, a chimeric antigen receptor T-cell (CAR-T) therapy, or a chimeric antigen receptor for phagocytosis (CAR-P) therapy, which targets at least one cancer-associated antigen and/or cancer-specific antigen. In some embodiments, the targeted cancer therapy is a therapy that targets a cancer-associated antigen. In some embodiments, the targeted cancer therapy is a therapy that targets a cancer-specific antigen. A skilled artisan will understand that cancer-associated antigens include a molecule, such as e.g., protein, present on cancer cells and on normal cells, or on many normal cells, but at much lower concentration than on cancer cells. In contrast, cancer-specific antigens generally include a molecule, such as e.g., protein which is present on cancer cells but absent from normal cells.

In some particular embodiments, provided herein are methods for activating integrin signaling in order to overcome CD47 checkpoint inhibition. In some embodiments, the disclosed methods further promote macrophage phagocytic signaling pathway. Some embodiments of the disclosure also provide methods for treatment of cancer, including solid tumor and hematologic malignancy, in an individual by promoting macrophage-mediated engulfment of cancer cells. In certain embodiments, the use of integrin activation in combination with one or more targeted cancer therapy, such as antibody therapies, chimeric antigen receptor T-cell (CAR-T) therapies, chimeric antigen receptor for phagocytosis (CAR-P) therapies, adoptive transfer macrophages is provided.

Currently, antibody blockade is used to eliminate the CD47 “Don't eat me” signal. The cancer therapy strategy disclosed herein relies on relieving an inhibitory checkpoint (CD47) that prevents macrophage activation. As described in more detail below, the experimental data presented herein has demonstrated that small molecule activators of integrin also overcome the inhibitory CD47 signal. This alternate strategy may be more cost effective.

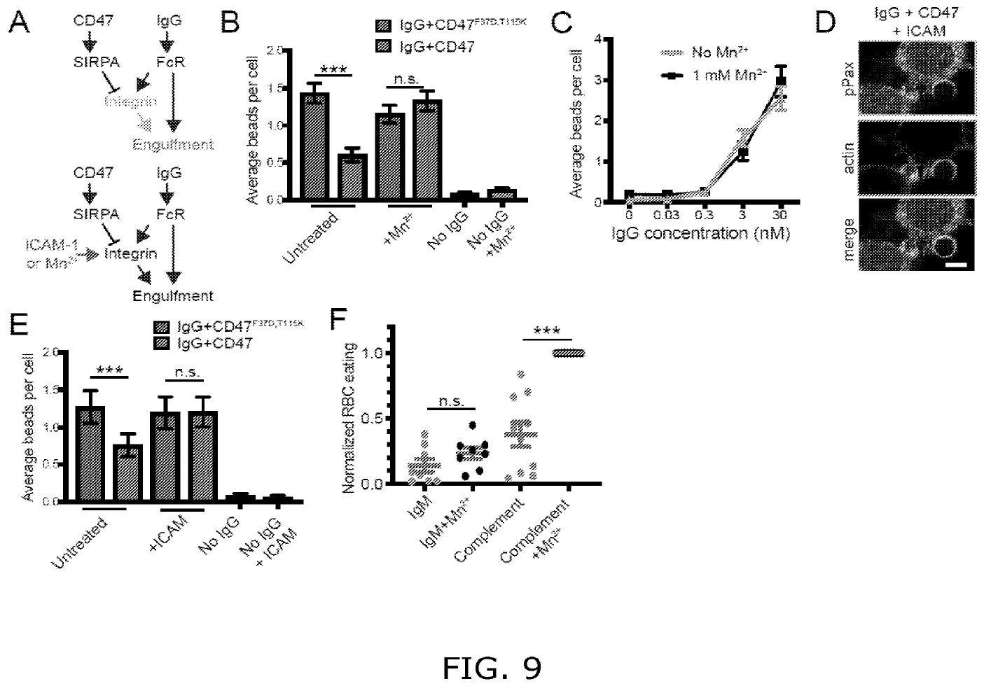

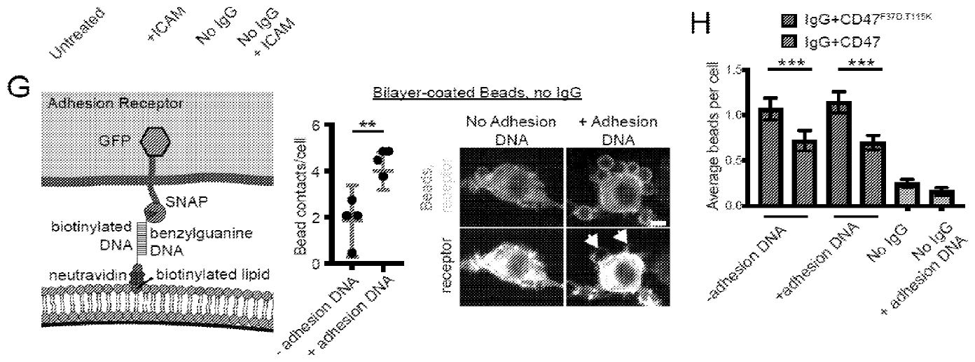

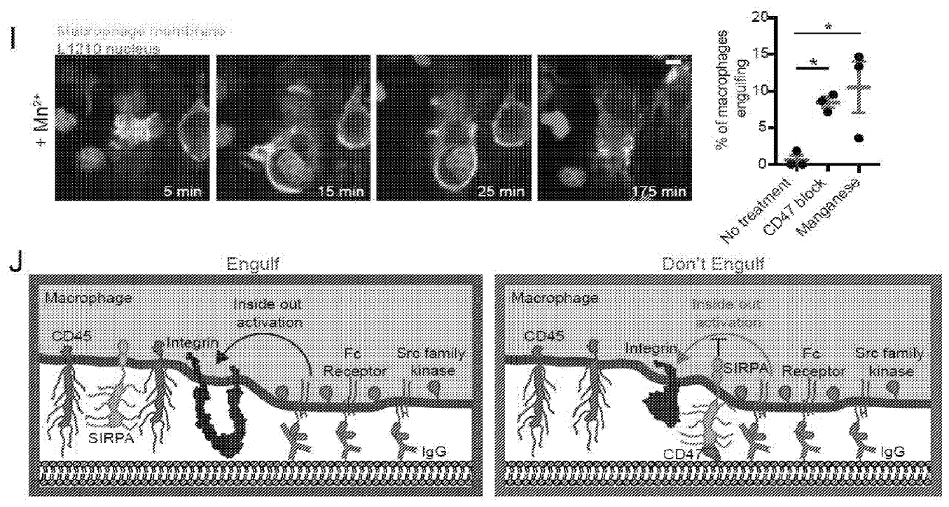

As described in greater detail below, the experimental data presented herein has demonstrated that activation of integrin signaling using manganese or the high-affinity integrin ligand ICAM-1 bypasses the inhibitory CD47 checkpoint and allows macrophages to engulf whole cancer cells. Moreover, experimental data presented in the Examples section has further demonstrated that integrin activation is equally effective to a CD47 function blocking antibody. Furthermore, experimental data presented in the Examples section also has shown that an integrin agonist that activates integrin allows macrophages to overcome the CD47 inhibitory checkpoint and engulf whole cancer cells. Hence, without being bound to any particular theory, integrin agonists may be administered as a cancer treatment, promoting macrophage-mediated engulfment of whole cancer cells. In particular, as shown in FIG. 9F, integrin activation via Mn2⁺ also promotes engulfment of complement-opsonized red blood cells (RBCs), presumably because this bypasses CD47. In addition, the experimental data presented herein also indicated that integrin activation also augments engulfment of CD47⁺ complement⁺ targets. As macrophages are adept at penetrating the tumor microenvironment, this would be a viable strategy for treating any solid or hematological malignancy.

Integrin activation has been previously shown to shrink some tumors, but the mechanism for this activity has not been elucidated. In the present disclosure, Experimental data presented in the Examples section has shown that integrin activation can overcome CD47 signaling, which demonstrates that patients with elevated levels of CD47 would likely benefit from treatment with an integrin agonist.

In particular, the experimental data described herein suggests that patients that are determined or predicted to be good candidates for treatment with a CD47 function blocking antibody would also be good candidates for treatment with an integrin agonist. Without being bound to any particular theory, it is believed that integrin activation could be used in combination with adoptive transfer of macrophages and/or dendritic cells that have been engineered to increase engulfment of cancer cells. For example, engulfment of cancer cells by macrophages can directly shrink the tumor, because the macrophages eat and digest the cancer cells. Additionally, phagocytosis in response to CD47/SIRPα-blocking agents results in antigen uptake and cross-presentation, thereby stimulating the adaptive immune systems. CD47/SIRPα blocking therapies may therefore synergize with immune checkpoint inhibitors that target the adaptive immune system. As a critical regulator of macrophage phagocytosis and activation, the potential applications of CD47/SIRPα blocking therapies extend beyond human cancer. They may be useful for the treatment of infectious disease, conditioning for stem cell transplant, and many other clinical indications such as, e.g., atherosclerosis. Indeed, immunotherapy involving CD47-blocking antibodies has been shown to restore phagocytosis and prevent atherosclerosis, which is a disease process that underlies heart attack and stroke. Atherogenesis is associated with upregulation of CD47, a key anti-phagocytic molecule that is known to render malignant cells resistant to programmed cell removal, or “efferocytosis.” Administration of CD47-blocking antibodies was found to reverse this defect in efferocytosis, normalize the clearance of diseased vascular tissue, and ameliorate atherosclerosis in multiple mouse models. Further information in this regard can be found in, e.g., Kojima et al., (Nature, 536:86-90, 2016), which is hereby incorporated by reference.

It is contemplated that the methods for activating integrin as disclosed herein could be deployed as a macrophage boosting strategy which synergizes with traditional chemotherapies. Without being bound to any particular theory, it is believe that the macrophages or dendritic cells can more easily take up dead cells or damaged cancer cells and subsequently present the cancer antigen to the adaptive immune system.

### Definitions

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

The terms “administration” and “administering”, as used herein, refer to the delivery of a bioactive composition or formulation by an administration route including, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

The term “agonist” is art-recognized, and refers to an agent or compound that binds to a target polypeptide (e.g., integrin) and stimulates, increases, activates, facilitates, enhances activation, sensitizes, or up-regulates the activity of the target polypeptide. As such, the term includes compounds and compositions that enhance or promote a function or activity (such as integrin binding to its ligand or conversion of integrin from inactive state to active state or phosphorylation of an intracellular protein). The term “cancer” refer to the presence of cells possessing characteristics typical of cancer-causing cells, such as uncontrolled proliferation, immortality, metastatic potential, rapid growth and proliferation rate, and certain characteristic morphological features. Cancer cells are often observed aggregated into a tumor, but such cells can exist alone within an animal subject, or can be a non-tumorigenic cancer cell, such as a leukemia cell. Thus, the terms “cancer” or can encompass reference to a solid tumor, a soft tissue tumor, or a metastatic lesion. As used herein, the term “cancer” includes premalignant, as well as malignant cancers. In some embodiments, the cancer is a solid tumor, a soft tissue tumor, or a metastatic lesion.

As used herein, and unless otherwise specified, a “therapeutically effective amount” of an agent is an amount sufficient to provide a therapeutic benefit in the treatment or management of the cancer, or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, an “individual” or a “subject” includes animals, such as human (e.g., human subjects) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, e.g., sheep, dogs, cows, chickens, amphibians, reptiles, etc.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is understood that aspects and embodiments of the disclosure described herein include “comprising,” “consisting,” and “consisting essentially of” aspects and embodiments. As used herein, “comprising” is synonymous with “including,” “containing,” or “characterized by,” and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or step.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Headings, e.g., (a), (b), (i) etc., are presented merely for ease of reading the specification and claims. The use of headings in the specification or claims does not require the steps or elements be performed in alphabetical or numerical order or the order in which they are presented.

### Integrins

Integrins are non-covalently linked α/β heterodimeric receptors that mediate cell adhesion, migration and signaling. Together with their ligands, integrins play central roles in many processes including development, hemostasis, inflammation and immunity, and in pathologic conditions such as cancer invasion and cardiovascular disease. Key leukocyte functions, such as activation, migration, tissue recruitment and phagocytosis, are essential for their normal immune response to injury and infection and in various conditions, including inflammatory and autoimmune disorders. The β2 (b2) integrins, a sub-family of α/β heterodimeric integrin receptors have a common β-subunit (β2, CD18) but distinct α-subunits (CD11a, CD11b, CD11c and CD11d. They regulate leukocyte functions, including via highly expressed integrins CD11a/CD18 (also known as LFA-1) and CD11b/CD18 (also known as Mac-1, CR3 and αMβ2) [2] that recognize a variety of ligands. For example, CD11b/CD18 recognizes >30 ligands, including the complement fragment iC3b, Fibrinogen, CD40L and ICAM-1 as ligands, among various others. CD11b/CD18 has been implicated in many inflammatory and autoimmune diseases. These include ischemia-reperfusion injury (including acute renal failure and atherosclerosis), multiple sclerosis (MS), tissue damage, transplantation, lupus, lupus nephritis, macular degeneration, glaucoma, stroke, neointimal thickening in response to vascular injury and the resolution of inflammatory processes. Further information in this regard can be found in, e.g., a recent review by Takada et al. (Genome Biology, 8:215, 2007), which is hereby incorporated by reference.

### CD47 and SIRPα Axis

CD47 is a cell surface molecule implicated in cell migration and T cell and dendritic cell activation. In addition, CD47 functions as an inhibitor of phagocytosis through ligation of signal-regulatory protein alpha (SIRPα, also referred to herein as SIRPA) expressed on phagocytes, leading to tyrosine phosphatase activation and inhibition of myosin accumulation at the submembrane assembly site of the phagocytic synapse. As a result, CD47 conveys a “don't eat me signal”. Loss of CD47 leads to homeostatic phagocytosis of aged or damaged cells.

CD47 polypeptide is comprised of an extracellular IgV set domain, a 5 membrane spanning transmembrane domain, and a cytoplasmic tail that is alternatively spliced. Two ligands bind CD47: thrombospondin-1 (TSP1), and signal inhibitory receptor protein alpha (SIRPα). TSP1 binding to CD47 activates the heterotrimeric G protein Gi, which leads to suppression of intracellular cyclic AMP (cAMP) levels. In addition, the TSP1-CD47 pathway opposes the beneficial effects of the nitric oxide pathway in all vascular cells. Elevated levels of CD47 expression are observed on multiple human tumor types, allowing tumors to escape the innate immune system through evasion of phagocytosis. This process occurs through binding of CD47 on tumor cells to SIRPα on phagocytes, thus promoting inhibition of phagocytosis and tumor survival.

SIRPα is expressed on various cell types, such as hematopoietic cells, including macrophages and dendritic cells. When it engages CD47 on a potential phagocytic target cell, phagocytosis is slowed or prevented. The CD47-SIRPα interaction effectively sends a “don't eat me” signal to the phagocyte. Thus, blocking the SIRPα-CD47 interaction with a monoclonal antibody in this therapeutic context has been shown to provide an effective anti-cancer therapy by promoting, i.e., increasing, the uptake and clearance of cancer cells by the host's immune system by increasing phagocytosis. This mechanism has been shown to be effective in leukemias, lymphomas, and many types of solid tumors.

Various anti-CD47 antibodies have demonstrated pre-clinical activity against many different human cancers both in vitro and in mouse xenotransplantation models. In addition to CD47, SIRPα can also be targeted as a therapeutic strategy; for example, anti-SIRPα antibodies administered in vitro caused phagocytosis of tumor cells by macrophages.

### Innate Immune System and CD47

The innate immune system is finely balanced to rapidly activate in response to pathogenic stimuli, but remain quiescent in healthy tissue. Macrophages, key effectors of the innate immune system, measure activating and inhibitory signals to set a threshold for engulfment and cytokine secretion. The cell surface protein CD47 is a “Don't Eat Me” signal that protects healthy cells from macrophage engulfment (Oldenborg et al., 2000). Hematopoietic cells lacking CD47 are rapidly engulfed by macrophages and trigger dendritic cell activation (Oldenborg et al., 2000; Yi et al., 2015). CD47 also functions in the nervous system, protecting active synapses from pruning by microglia (Lehrman et al., 2018). CD47 expression is often increased on cancer cells as a mechanism to evade immune detection (Chao et al., 2012; Jaiswal et al., 2009; Majeti et al., 2009; Oldenborg et al., 2001, 2000). CD47 function-blocking antibodies result in decreased cancer growth or tumor elimination (Advani et al., 2018; Chao et al., 2010a; Gholamin et al., 2017; Jaiswal et al., 2009; Willingham et al., 2012). Augmenting macrophage function by CD47 blockade may also be beneficial in other disease contexts, like atherosclerosis or viral infection (Chain et al., 2020; Kojima et al., 2016). Despite the therapeutic promise of manipulating CD47 signaling, the mechanism by which CD47 suppresses macrophage engulfment remains unclear.

CD47 on the surface of target cells is recognized by SIRPA (Signal Regulatory Protein α) on macrophages or dendritic cells (Jiang et al., 1999; Liu et al., 2015; Okazawa et al., 2005; Oldenborg et al., 2000; Seiffert et al., 1999; Tseng et al., 2013; Yi et al., 2015). SIRPA is an inhibitory receptor containing multiple intracellular Immune Tyrosine-based Inhibitory Motifs (ITIMs; Kharitonenkov et al., 1997). Macrophages lacking SIRPA do not exhibit reduced phagocytosis of CD47-bearing targets, suggesting that SIRPA is the primary transducer of the CD47 signal (Okazawa et al., 2005; Oldenborg et al., 2000). Activation of the inhibitory receptor SIRPA must be controlled with high fidelity to suppress engulfment of viable cells when CD47 is present while allowing for robust engulfment of targets lacking CD47. CD47 binding triggers SIRPA phosphorylation by Src family kinases (Barclay and Brown, 2006), but how CD47 binding is translated across the cell membrane to drive SIRPA phosphorylation is not known. Phosphorylated SIRPA recruits the phosphatases SHP-1 and SHP-2 (Fujioka et al., 1996; Noguchi et al., 1996; Okazawa et al., 2005; Oldenborg et al., 2001; Veillette et al., 1998), but the downstream targets of these phosphatases and their relationship to the engulfment process also remain unclear.

In vivo, CD47 suppresses multiple different pro-engulfment “Eat Me” signals, including IgG, complement and calreticulin (Chen et al., 2017; Gardai et al., 2005; Oldenborg et al., 2001). This complexity, in addition to substantial variation in target size, shape and concentration of “Eat Me” signals, can make a quantitative, biochemical understanding of receptor activation difficult. To overcome this problem, some experiments described in the Examples section below have been designed to utilize a synthetic target cell-mimic with a defined set of signals to interrogate the mechanism of SIRPA activation and its downstream targets. As described in more detail below, results from experimental data presented in the Examples section have demonstrated that CD47 ligation altered SIRPA localization, positioning SIRPA for activation at the phagocytic synapse. At the phagocytic synapse, SIRPA inhibited integrin activation to limit macrophage spreading across the surface of the engulfment target. Directly activating integrin eliminated the effect of CD47 and rescued engulfment, similar to the effect of a CD47 function-blocking antibody. Thus, the CD47-SIRPA axis suppresses phagocytosis by inhibiting inside-out activation of integrin signaling in the macrophage, with implications to cancer immunotherapy applications.

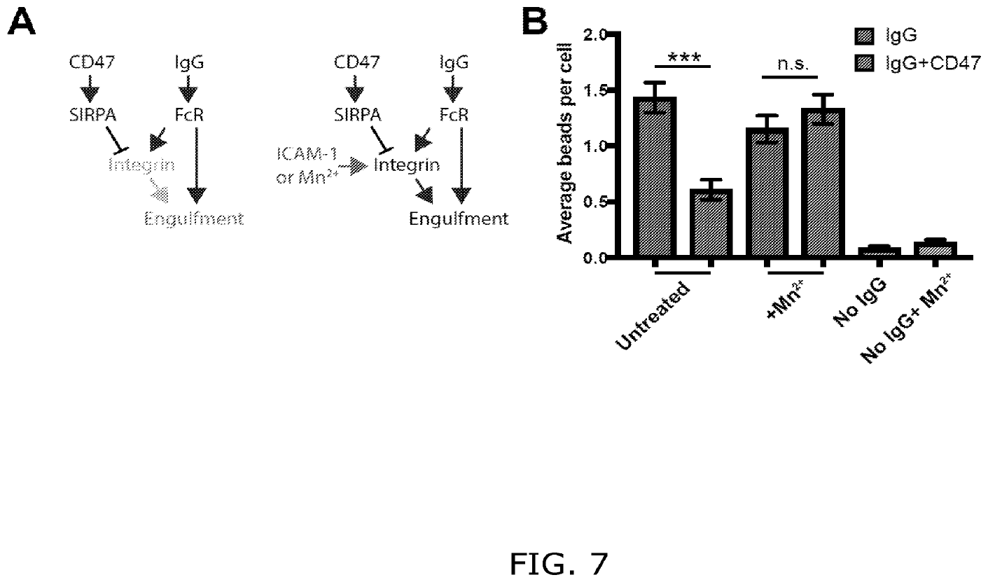

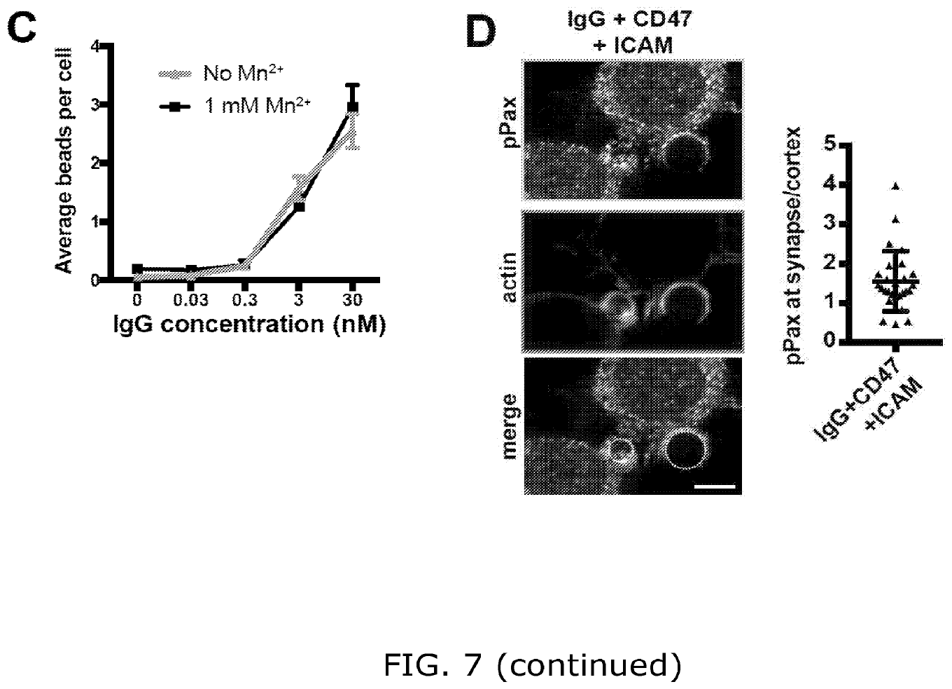

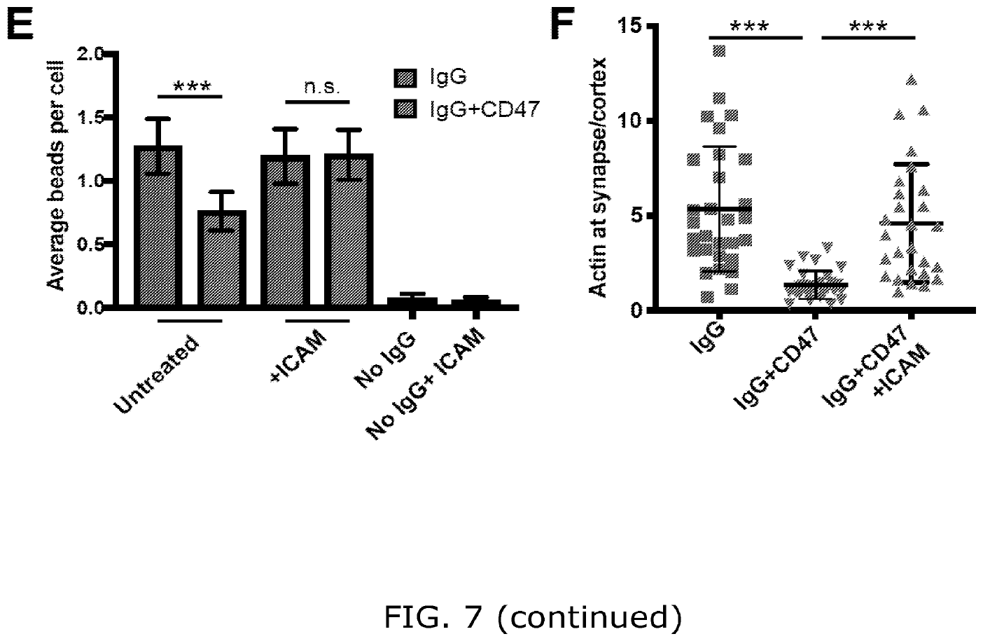

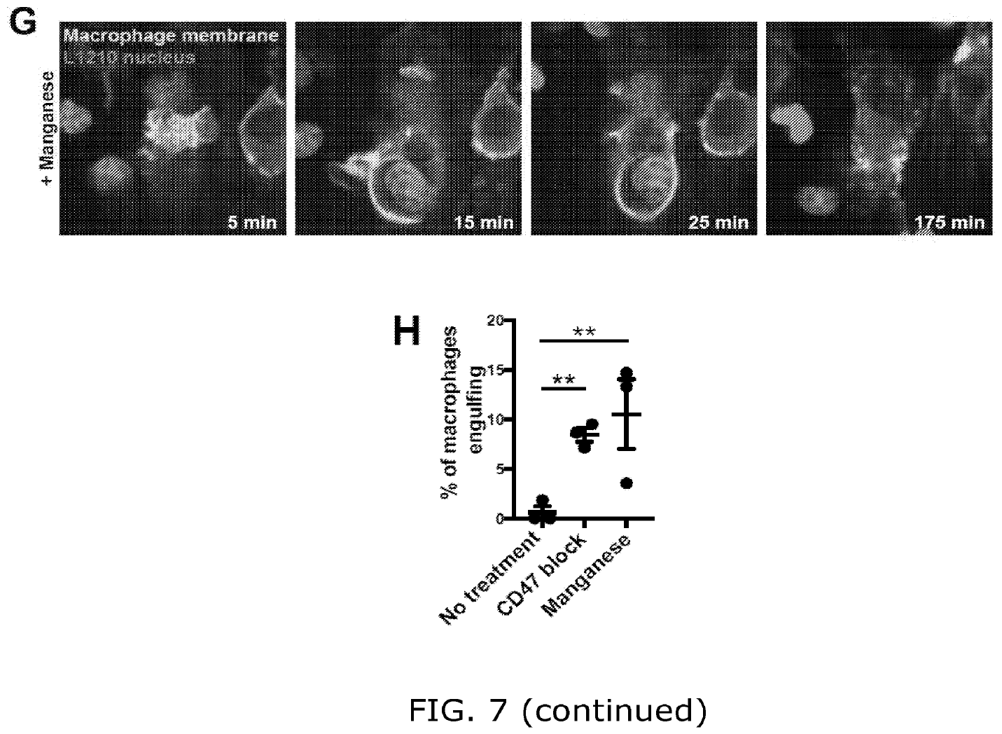

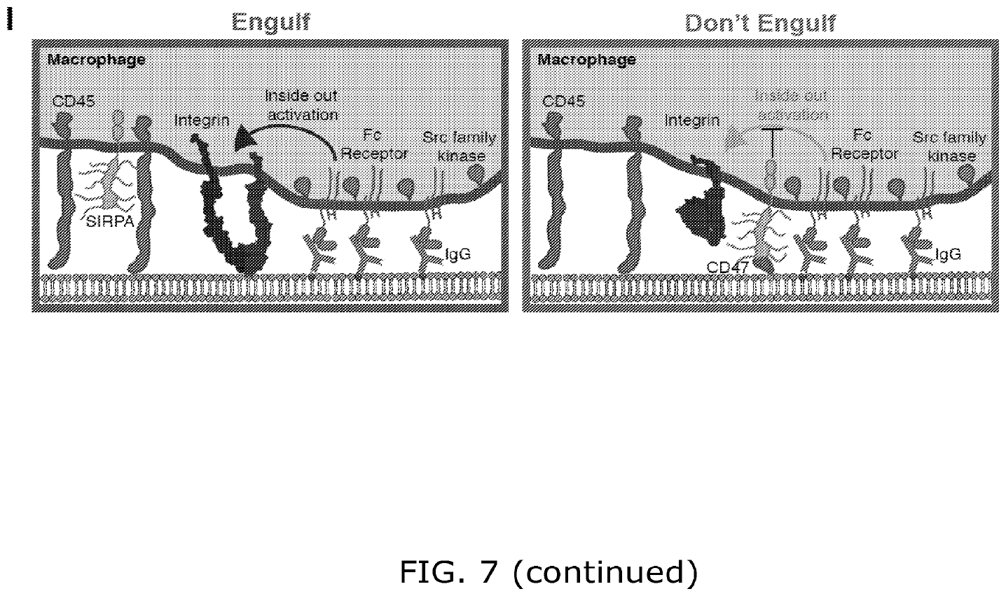

CD47-SIRPA signaling suppresses engulfment, protecting viable cells and allowing cancer cells to evade the innate immune system (Jaiswal et al., 2009; Majeti et al., 2009; Oldenborg et al., 2000). Although CD47 blockade is a promising new target for cancer therapies (Advani et al., 2018; Gholamin et al., 2017; Willingham et al., 2012), the mechanism of CD47-SIRPA signaling has not been clarified. As described in more detail below, experimental data presented in the Examples section has indicated that CD47 dampened IgG-mediated phagocytosis but this suppressive effect could be overcome by a surplus of IgG. Mechanistically, experimental data presented in the Examples section has demonstrated that localizing SIRPA to the phagocytic synapse was sufficient to activate this inhibitory receptor. Once active, SIRPA suppressed engulfment by preventing integrin activation (FIG. 7I).

The experimental results described herein, e.g., in the Examples section, demonstrate that SIRPA localization is a key determinant of its activity. In the absence of CD47, SIRPA is relegated to the phosphatase-rich zone outside the cell bead interface (Freeman et al., 2016; Goodridge et al., 2011). This localization prevents SIRPA activation. Conversely, CD47 binding retained SIRPA at the Src-kinase rich phagocytic cup, where it is activated and suppresses engulfment. Spatial segregation of Src-family kinase activity at the central phagocytic synapse and CD45 phosphatase activity at the periphery underlies the activation of many activating receptors (TCR, Fc Receptor, (Freeman et al., 2016; James and Vale, 2012). The experimental results described herein expands this model, suggesting that exclusion of inhibitory receptors like SIRPA may be a pre-requisite for efficient engulfment. Further, these data suggest a new paradigm for regulating inhibitory receptors based on conditional recruitment to the immunological synapse.

SIRPA exclusion from the phagocytic synapse in the absence of CD47 prevents basal inhibition of engulfment and allows positive signaling to dominate. SIRPA may be sterically excluded from the phagocytic synapse based on the size of its bulky extracellular domain, as replacing the extracellular domain with a small, inert protein (FRB) allowed SIRPA to enter the phagocytic synapse. Without being bound to any particular theory, it was hypothesized that the bulky SIRPA extracellular domain may be sterically excluded from the phagocytic synapse based on height alone. The FcR-IgG complex is ˜11.5 nm tall (Lu et al., 2011), and the data presented herein demonstrate that both unligated SIRPA and CD47-bound SIRPA are excluded from these receptor-ligand clusters. Between IgG clusters, integrin forms a diffusion barrier in the phagocytic synapse that prevents bulky proteins from entering (Freeman et al., 2016). While extended integrin is quite tall, engaged integrin is tilted and has been shown to drive exclusion of the bulky transmembrane phosphatase CD45 (Freeman et al., 2016; Swaminathan et al., 2017). Although aglycosylated CD45 is larger than SIRPA (17 nm and 12 nm respectively), the size of both extracellular domains is increased by extensive glycosylation (Chang et al., 2016; Hatherley et al., 2008). Thus, steric exclusion may be sufficient to explain the depletion of SIRPA at the immunological synapse. To support this hypothesis, experimental data presented herein has demonstrated that shortening the distance between the macrophage and its target increased SIRPA exclusion. CD47 binding may also alter SIRPA localization. Biophysical studies show that unligated proteins that are the same size or even slightly smaller than the height of a cell-cell synapse are excluded from the synapse (Schmid et al., 2016). Ligand binding is sufficient to drive synapse localization (Schmid et al., 2016). Thus, SIRPA may be sterically excluded unless CD47 ligation overcomes the energetic barrier preventing SIRPA from entering the immunological synapse. While the data presented herein demonstrates that SIRPA exclusion can be driven by altering the height of the immunological synapse, contribution of other exclusion mechanisms, such as lateral crowding or interactions with the surrounding glycocalyx, is also possible.

After addressing the mechanism of SIRPA activation, additional experiments were performed to identify the targets of CD47-SIRPA signaling. Previous work shows that SIRPA activation dramatically reduces global phosphotyrosine, including phosphorylation of mDia, paxillin, talin, alpha-actinin and non-muscle myosin IIA (Okazawa et al., 2005; Tsai and Discher, 2008). However, discerning between direct targets of SIRPA-bound phosphatases and indirect targets resulting from an upstream block in the engulfment signaling cascade has been challenging. Because blocking non-muscle myosin II decreases phagocytosis to a similar extent as CD47, myosin has been presumed to be the primary target of SIRPA, suggesting a model where CD47 inhibits “pulling” of the phagocytic target into the macrophage (Chao et al., 2012; Tsai and Discher, 2008). However, experimental data presented herein has demonstrated that the inhibitory effect of CD47-SIRPA can be eliminated by re-activating integrin, indicating that the direct targets of SIRPA-bound SHP phosphatases are upstream of integrin activation. Instead of a pulling model, it was hypothesized that CD47 inhibits spreading of the macrophage around the phagocytic target. Experimental data presented herein indicate that blocking αMβ2 integrin had the largest effect on engulfment. However, instead of targeting a specific integrin subset directly, it was further hypothesized that SIRPA bound phosphatases deactivate an upstream step in the inside-out activation signaling pathway or an integrin regulator. SHP-2 has previously been shown to directly dephosphorylate Fak (Yu et al., 1998) and vinculin (Campbell et al., 2018), thus SHP-2 may act upon these key integrin regulators. However, given the broad specificity of SHP-1 and SHP-2, these phosphatases may dephosphorylate several targets at the phagocytic cup to suppress signaling.

Experimental data presented herein illustrate that CD47-SIRPA prevents integrin activation, allowing macrophages to quickly discriminate between targets based on the presence of CD47. In addition to immediately inactivating integrin to prevent engulfment of a CD47-positive cell, SIRPA may also contribute to a long-term transcriptional down regulation of integrins (Liu et al., 2008). While this decrease in integrin expression does not explain how SIRPA prevents phagocytosis specifically of CD47-bound targets, it indicates that long-term exposure to activated SIRPA may decrease overall phagocytic capacity. Paradoxically, SIRPA may also be required for integrin-dependent cell migration, as fibroblasts lacking SIRPA have impaired motility (Alenghat et al., 2012; Inagaki et al., 2000; Motegi et al., 2003). In this context, SIRPA may promote integrin turnover to provide the dynamic interactions necessary for motility.

By suppressing integrin activation, CD47-SIRPA signaling may be able to suppress many different signaling pathways. CD47 has been reported to affect dendritic cell activation, cancer cell killing via a nibbling behavior (called trogocytosis), and complement-mediated engulfment (Caron et al., 2000; Matlung et al., 2018; Oldenborg et al., 2001; Tamada et al., 2004; Wu et al., 2018; Yi et al., 2015). These processes are triggered by diverse positive signaling receptors, but all require inside-out activation of integrin (Caron et al., 2000; Matlung et al., 2018; Oldenborg et al., 2001; Tamada et al., 2004; Wu et al., 2018; Yi et al., 2015). Targeting integrin, a common co-receptor, may explain how CD47-SIRPA signaling can regulate these diverse processes.

In addition, experimental data presented in the Examples section has shown that integrin activation by manganese can drive engulfment of whole cancer cells by bone marrow derived macrophages. As a cancer treatment, CD47 blockade synergizes with therapeutic antibodies, like rituximab (Advani et al., 2018; Chao et al., 2010a). Activating integrins with a small molecule agonist in combination with antibody therapeutics may have a similar synergistic effect as CD47 blockade.

Small molecule agonists of the αM integrin subunit drive tumor regression in a macrophage-dependent manner (Panni et al., 2019; Schmid et al., 2018). Experimental data presented in the Examples section below indicates that these small molecules may promote tumor regression partially by allowing macrophages to bypass the CD47 inhibitory signal.

### Methods of the Disclosure

In one aspect, some embodiments of the disclosure relate to a method for treatment of a cancer in an individual in need thereof. The methods include administering to the individual (a) therapeutically effective amount of an integrin agonist; and (b) a cancer therapy that targets at least one cancer-associated antigen and/or cancer-specific antigen.

The term “agonist” is art-recognized, and refers to an agent or compound that binds to a target polypeptide (e.g., integrin) and stimulates, increases, activates, facilitates, enhances activation, sensitizes, or up-regulates the activity of the target polypeptide. As such, the term includes compounds and compositions that enhance or promote a function or activity (such as integrin binding to its ligand or conversion of integrin from inactive state to active state or phosphorylation of an intracellular protein). In some embodiments of the disclosure, the integrin agonist activates one or more α integrins and/or β integrins. In some embodiments, the integrin agonist activates one or more α integrins. In some embodiments, the integrin agonist activates one or more β integrins. In some embodiments, the integrin agonist activates a combination of integrins and β integrins. In some embodiments, the integrin agonist activates αVβ3, αLβ2, αMβ2, αXβ2, αDβ2, α4β1, α4β7, αEβ7, or a combination of any thereof. In some embodiments, the integrin agonist activates αVβ3 integrin. In some embodiments, the integrin agonist activates β2 integrin.

Generally, integrins can be activated by any one of techniques and strategies known in the art, including manganese treatment, binding of high affinity integrin ligands, and small molecule agonists. Accordingly, in some embodiments of the disclosed methods, the integrin agonist includes a manganese treatment, a high affinity integrin ligand, a small molecule agonist, or a combination of any thereof. In some embodiments, the small molecule agonist of integrin includes leukadherin-1 (LA1), ADH-503, or a combination thereof. Additional small molecule agonists of integrin suitable for the methods disclosed herein include those describes in U.S. Pat. No. 10,287,264 and PCT Patent Publication No. WO2016/176400A2.

In some embodiments, integrins can be activated by a high affinity integrin ligand. Further information in this regard can be found in, for example, in a recent review by Banno et al. (Biochem Soc Trans. 36: 229-234, April 2008). Generally, the high affinity integrin ligand can be any high affinity integrin ligand known in the art and can be, for example, ICAM-1, ICAM-2, ICAM-3, VCAM-1, MAdCAM-1, E-cadherin, JAM-1, JAM-2, JAM-3. Accordingly, in some embodiments of the disclosed methods, the integrin agonist includes one or more of ICAM-1, ICAM-2, ICAM-3, VCAM-1, MAdCAM-1, E-cadherin, JAM-1, JAM-2, JAM-3, and combinations of any thereof. In some embodiments, the integrin agonist includes ICAM-1.

This discussed above, the methods disclosed herein are suitable the treatment of cancer, which generally refers to a disease characterized by the rapid and uncontrolled growth of aberrant cells. The aberrant cells may form solid tumors or constitute a hematological malignancy. Cancer cells can spread locally or through the bloodstream and lymphatic system to other parts of the body. There are no specific limitations with respect to the cancers which can be treated by the methods of the present disclosure.

Non-limiting examples of suitable cancers include ovarian cancer, renal cancer, breast cancer, prostate cancer, liver cancer, brain cancer, lymphoma, leukemia, cervical cancer, skin cancer, pancreatic cancer, colorectal cancer, lung cancer and the like. Other cancers that can be suitable treated with the compositions and methods of the present disclosure include, but are not limited to, AML, ALL, CML, adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, transitional cell carcinoma, vaginal cancer, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, gallbladder cancer, gastrointestinal carcinoid tumors, gastrointestinal stromal tumors, gestational trophoblastic disease, Hodgkin's lymphoma, Kaposi's sarcoma, kidney cancer, laryngeal and hypopharyngeal cancer, liver cancer, lung cancer, lung carcinoid tumors, brain cancers, central nervous system (CNS) cancers, peripheral nervous system (PNS) cancers, breast cancer, cervical cancer, childhood Non-Hodgkin's lymphoma, colon and rectum cancer, Non-Hodgkin's lymphoma, malignant mesothelioma, multiple myeloma, myelodysplastic syndrome, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g., uterine sarcoma), transitional cell carcinoma, vaginal cancer, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g., uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia. Particularly suitable cancers include, but are not limited to, breast cancer, ovarian cancer, lung cancer, pancreatic cancer, mesothelioma, leukemia, lymphoma, brain cancer, prostate cancer, multiple myeloma, melanoma, bladder cancer, bone sarcomas, soft tissue sarcomas, retinoblastoma, renal tumors, neuroblastoma, and carcinomas.

Accordingly, in some embodiments, the cancer is a solid tumor. In some embodiments, the cancer is a non-solid tumor. In some embodiments, the cancer is a hematologic malignancy. In some embodiments, the cancer is selected from the group consisting of leukemia, pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, lymphoma, and glioblastoma.

The term “cancer therapy” should herein be understood in its general sense and refers to any therapy useful in treating cancer. Examples of anti-cancer therapeutic agents include, but are not limited to, e.g., surgery, chemotherapeutic agents, immunotherapy, growth inhibitory agents, cytotoxic agents, agents used in radiation therapy, anti-angiogenesis agents, apoptotic agents, anti-tubulin agents, and other agents to treat cancer. Similarly, the term “tumor therapy” should herein be understood in its general sense, i.e. that the objective is to significantly reduce the size the tumors within the organism. In some embodiments, the tumors are completely eliminated. In some embodiments, the targeted cancer therapy includes an antibody therapy, a chimeric antigen receptor T-cell (CAR-T) therapy, a chimeric antigen receptor for phagocytosis (CAR-P) therapy, a myeloid-targeting therapy, or a combination thereof, that targets at least one cancer-associated antigen and/or cancer-specific antigen. In some embodiments, the targeted cancer therapy includes an antibody, a CAR-T, or a CAR-P that binds to a cell surface-associated antigen expressed on the cancer cell. In some embodiments, the targeted cancer therapy includes a therapy targeting myeloid cells. In some embodiments, the targeted cancer therapy includes adoptive transfer of immune cells expressing a CAR-P. In some embodiments, the immune cells expressing a CAR-P include macrophages, dendritic cells, natural killer cells, neutrophils, or a combination of any thereof.

In some embodiments, the targeted cancer therapy includes one or more macrophages expressing a CAR-P which includes an intracellular signaling domain from an engulfment receptor. In some embodiments, the intracellular signaling domain of the engulfment receptor comprises at least 1, at least 2, at least 3, at least 4, or at least 5 ITAM motifs. In some embodiments, the intracellular signaling domain from the engulfment receptor is capable of mediating endogenous phagocytic signaling pathway. In some embodiments, engulfment receptor is selected from the group consisting of Megf10, FcRγ, Bai1, MerTK, TIM4, Stabilin-1, Stabilin-2, RAGE, CD300f, Integrin subunit αv, Integrin subunit β5, CD36, LRP1, SCARF, C1Qa, and Ax1. In some embodiments, the one or more phagocytic cells is selected from the group consisting of macrophages, dendritic cells, mast cells, monocytes, neutrophils, microglia, and astrocytes. In some embodiments, at least one of the one or more phagocytic cells is a bone marrow derived macrophage (BMDM) or a bone marrow derived dendritic cell (BMDC). In some embodiments, the phagocytic cell is a J774A.1 macrophage. In some embodiments, the phagocytic cell is an AW264.7 macrophage. In some embodiments, the phagocytic cell is a Bone marrow derived macrophages (BMDM) were generated from the hips and long bones of C57BL/6J mice. These cells are available from many sources, including the American Type Culture Collection (Manassas, Va.). In some embodiments, the phagocytic cell is derived from the same individual having cancer, where phagocytes are removed from an individual (blood, tumor or ascites fluid), and modified so that they express the CAR-P receptors specific to a particular form of antigen associated with the individual's cancer. Additional information regarding compositions and methods related to CAR-P technology can be found in, e.g., PCT Patent Publication No. WO2020097193A1, which is hereby incorporated by reference in its entirety.

In some embodiments, the targeted cancer therapy is an antibody therapy including an anti-CD47 antibody. In some embodiments, the anti-CD47 antibody is a CD47 function-blocking antibody, e.g., anti-CD47 antibody which binds to CD47 and antagonize the interaction with SIRPα. By blocking that interaction, and because of the Fc region of the antibody, the effect of the CD47 antibodies can be similar to the effect of the SIRPα-based drugs. Exemplary CD47 antibodies are described in the literature such as Celgene's WO2016/109415; Chugai's US2008/0107654; InhibRx WO2013/119714; Janssen's WO2016/081423, and Stanford's WO2009/091601. Because these antibodies bind red blood cells, a dosing regimen that takes this into account has been developed and is described in WO2014/149477. The properties of a useful anti-CD47 antibody include simply the ability to bind to CD47 in a way that ultimately inhibits signaling by SIRPα, i.e., as an antagonist. Additional CD47 antibodies suitable for the methods disclosed herein include, but are not limited to, those described in WO2014/093678; US2014/0161805; US2014/0161825; US2012/0189625; US2012/0156724; US2013/0142786; and US2019/0135921A1, all of which are hereby incorporated by reference. In some embodiments, the anti-CD47 antibody includes clone miap301 (BioLegend, Cat #127520).

In some embodiments, the targeted cancer therapy is an antibody therapy including an anti-SIRPα antibody. Exemplary anti-SIRPα antibodies suitable for the methods disclosed herein include those described in WO2015/138600A2, US2014/0242095A1, US2016/0333093A1, all of which are hereby incorporated by reference. In some embodiments, the targeted cancer therapy is an antibody therapy including a combination of anti-CD47 antibody and anti-SIRPα antibody.

In some embodiments, the targeted cancer therapy is a myeloid-targeting therapy, e.g., a therapy or treatment that targets myeloid cells, and/or is dependent on myeloid activity. Accordingly, in some embodiments, a myeloid-targeting therapy includes therapeutic agents that target non-myeloid cells (e.g., B cells by using a suitable agent such as Rituximab) but rely on myeloid cells to carry out at least part of their therapeutic effects. In some embodiments, a myeloid-targeting therapy targets myeloid cells, such as, e.g., monocytes, macrophages, dendritic cells, and granulocytes. Myeloid cells constitute a significant part of the immune system in the context of cancer, exhibiting both immuno-stimulatory effects, through their role as antigen presenting cells, and immuno-suppressive effects, through their polarization to myeloid-derived suppressor cells (MDSCs) and tumor-associated macrophages. While myeloid cells are rarely sufficient to generate potent anti-tumor effects on their own, they have the ability to interact with a variety of immune populations to aid in mounting an appropriate anti-tumor immune response. Generally, the myeloid-targeting therapy can be any myeloid-targeting therapy known in the art. Exemplary strategies suitable for targeting myeloid cells include, but are not limited to, (1) modulating the recruitment of MDSCs from peripheral blood; (2) promoting an immuno-stimulatory phenotype, primarily through maturation of myeloid precursors into inflammatory macrophages and antigen presenting dendritic cells (DCs); and (3) inhibiting the polarization of myeloid cells to MDSCs. In some embodiments, the myeloid-targeting therapy includes one or more antagonists of the CCL2/CCR2 axis, the CCL5/CCR5 axis, the CAF1/CSF1R axis, the CD47/SIRPα axis, or a combination of any thereof. Non-limiting examples of reagents suitable for a myeloid-targeting therapy include Carlumab, Plozalizumab, PF-04136309, Rituximab, NOX-E36, HuMax-IL8, BMS-986253, BMS-813160, Pexidartinib (PLX-3397), BLZ-945 and ARRY-382. Additional techniques, strategies, and reagents suitable for therapies targeting myeloid cells includes those described in Jahchan et al., (Front. Immunol. Jul. 25, 2019; e.g., Table 1) and Perry et al. (J. Exp. Med. March, 215 (3): 877, 2018; both of which are hereby incorporated by reference.

In principle, there are no specific limitations with respect to the procedures and techniques that can be suitably employed for delivery of the integrin agonists as described herein into the target cell. Non-limiting delivery procedures suitable for the methods disclosed herein include stable or transient transfection, lipofection, electroporation, microinjection, liposomes, iontophoresis, and infection with recombinant viral vectors. In some embodiments, the administration includes a viral-, particle-, liposome-, or exosome-based delivery procedure. In some embodiments, the administration includes delivering into endogenous cells ex vivo one or more integrin agonists as described herein. In some embodiments, the administration includes delivering into cells in vivo one or more integrin agonists as described herein.

Typically, a pharmaceutical composition is formulated to be compatible with its intended route of administration. The integrin agonists of the disclosure may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject integrin agonists of the disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such integrin agonists lies preferably within a range of circulating concentrations that include the ED₅₀ with little or no toxicity. The dosage may vary within this range depending upon the dosage form employed and the route of administration utilized. For any molecules or compositions used in the methods of the disclosure, the therapeutically effective dose can be estimated initially from cell culture assays. A dose may be formulated in animal models to achieve a circulating plasma concentration range that includes the IC50 (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

As discussed above, a “therapeutically effective amount or number” of a subject integrin agonist of the disclosure (e.g., an effective dosage) depends on the integrin agonist selected. For instance, single dose amounts in the range of approximately 0.001 to 0.1 mg/kg of patient body weight can be administered; in some embodiments, about 0.005, 0.01, 0.05 mg/kg may be administered. In some embodiments, 600,000 IU/kg is administered (IU can be determined by a lymphocyte proliferation bioassay and is expressed in International Units (IU) as established by the World Health Organization International Standard). The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the subject integrin agonist of the disclosure can include a single treatment or, can include a series of treatments. In some embodiments, the compositions are administered every 8 hours for five days, followed by a rest period of 2 to 14 days, e.g., 9 days, followed by an additional five days of administration every 8 hours.

In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation,

### Kits

In one aspect of the disclosure, provided herein are various kits for use in a method of treating a cancer as disclosed herein. The kits include (a) one or more integrin agonists and (b) instructions for use of the one or more integrin agonists in combination with a cancer therapy that targets a cancer-associated antigen and/or a cancer-specific antigen.

In some embodiments, the kits of the disclosure further include one or more syringes (including pre-filled syringes) and/or catheters (including pre-filled syringes) used to administer any one of the integrin agonists described herein a to a subject in need thereof. In some embodiments, a kit can have one or more additional therapeutic agents that can be administered simultaneously or sequentially with the other kit components for a desired purpose, e.g., for treating a cancer in a subject in need thereof.

For example, any of the above-described kits can further include one or more additional reagents, where such additional reagents can be selected from: dilution buffers; reconstitution solutions, wash buffers, control reagents, negative controls, and positive controls.

In some embodiments, the components of a kit can be in separate containers. In some other embodiments, the components of a kit can be combined in a single container.

In some embodiments, a kit of the disclosure can further include instructions for using the components of the kit to practice the methods. The instructions for practicing the methods are generally recorded on a suitable recording medium. For example, the instructions can be printed on a substrate, such as paper or plastic, etc. The instructions can be present in the kits as a package insert, in the labeling of the container of the kit or components thereof (i.e., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), are provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, N.Y.: Cold Spring Harbor Laboratory and Sambrook, J., & Russel, D. W. (2001). Molecular Cloning: A Laboratory Manual (3rd ed.). Cold Spring Harbor, N.Y.: Cold Spring Harbor Laboratory (jointly referred to herein as “Sambrook”); Ausubel, F. M. (1987). Current Protocols in Molecular Biology. New York, N.Y.: Wiley (including supplements through 2014); Bollag, D. M. et al. (1996). Protein Methods. New York, N.Y.: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, Calif.: Academic Press; Lefkovits, I. (1997). The Immunology Methods Manual: The Comprehensive Sourcebook of Techniques. San Diego, Calif.: Academic Press; Doyle, A. et al. (1998). Cell and Tissue Culture: Laboratory Procedures in Biotechnology. New York, N.Y.: Wiley; Mullis, K. B., Ferr6, F. & Gibbs, R. (1994). PCR: The Polymerase Chain Reaction. Boston: Birkhauser Publisher; Greenfield, E. A. (2014). Antibodies: A Laboratory Manual (2nd ed.). New York, N.Y.: Cold Spring Harbor Laboratory Press; Beaucage, S. L. et al. (2000). Current Protocols in Nucleic Acid Chemistry. New York, N.Y.: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1

### Additional Experimental Procedures

### Cell Cultures

RAW264.7 macrophages were provided by the ATCC and certified mycoplasma-free. The cells were cultured in DMEM (Gibco, Catalog #11965-092) supplemented with 1× Pen-Strep-Glutamine (Corning, Catalog #30-009 Cl) 1 mM sodium pyruvate (Gibco, Catalog #11360-070) and 10% heat inactivated fetal bovine serum (Atlanta Biologicals, Catalog #S11150H). To keep variation to a minimum, cells were discarded after 20 passages. L1210 cells were also acquired from the ATCC.

J774A.1 macrophages were provided by the UCSF cell culture facility. J774A.1 and 293T cells were tested for mycoplasma using the Lonza MycoAlert Detection Kit (Lonza, Catalog #LT07-318) and control set (Lonza, Catalog #LT07-518).

Bone marrow derived macrophages (BMDM) were generated from the hips and long bones of C57BL/6J mice as previously described (Weischenfeldt and Porse, 2008) except that purified 25 ng/ml M-CSF (Peprotech, Catalog #315-02) was used.

### Constructs and Antibodies

All relevant information regarding the constructs and antibodies discussed in the present disclosure is provided in the FIG. 9 (Key Resource Table), including a detailed description of the amino acid sequence of each construct and the catalog number of all antibodies.

### Lentivirus Production and Infection

All constructs were expressed in RAW264.7 using lentiviral infection. Lentivirus was produced in HEK293T cells transfected with pMD2.G (a gift from Didier Tronon, Addgene plasmid #12259 containing the VSV-G envelope protein), pCMV-dR8.91 (since replaced by second generation compatible pCMV-dR8.2, Addgene plasmid #8455), and a lentiviral backbone vector containing the construct of interest (derived from pHRSIN-CSGW, see STAR methods) using lipofectamine LTX (Invitrogen, Catalog #15338-100). Constructs are described in detail in the Key Resources Table. The media was harvested 72 hr post-transfection, filtered through a 0.45 μm filter and concentrated using LentiX (Takara Biosciences). After addition of the concentrated virus, cells were centrifuged at 2000×g for 45 min at 37° C. Cells were analyzed a minimum of 60 hr later, and maintained for a maximum of one week.

### Supported Lipid Bilayer Assembly

Preparation of Small Unilamellar Vesicles (SUV).

The following chloroform-suspended lipids were mixed and desiccated overnight to remove chloroform. 96.8% POPC (Avanti, Catalog #850457), 2% Ni²⁺-DGS-NTA (Avanti, Catalog #790404), 1% biotinyl cap PE (Avanti, Catalog #870273), 0.1% PEG5000-PE (Avanti, Catalog #880230, and 0.1% atto390-DOPE (ATTO-TEC GmbH, Catalog #AD 390-161). The lipid sheets were resuspended in PBS, pH7.2 (Gibco, Catalog #20012050) and stored under argon. The lipids were broken into small unilamellar vesicles via several rounds of freeze-thaws. The mixture was cleared using ultracentrifugation (TLA120.1 rotor, 35,000 rpm/53,227×g, 35 min, 4° C.). The lipids were then stored at 4° C. under argon for up to two weeks.

### Planar Bilayer Preparation for TIRF (Total Internal Reflection Fluorescence) Microscopy.

Ibidi coverslips (Catalog #10812) were RCA cleaned. Supported lipid bilayers were assembled in custom plasma cleaned PDMS (Dow Corning, catalog #3097366-0516 and 3097358-1004) chambers at room temperature for 1 hour. Bilayers were blocked with 0.2% casein (Sigma, Catalog #C5890) in PBS. Proteins were coupled to the bilayer for 45 min. Imaging was conducted in HEPES buffered saline (20 mM HEPES, 135 mM NaCl, 4 mM KCl, 10 mM glucose, 1 mM CaCl₂, 0.5 mM MgCl₂). Bilayers were assessed for mobility by either photobleaching or monitoring the mobility of single particles.

Bead Preparation.

8.6×10⁸ silica beads with a 5.02 μm diameter (10 μl of 10% solids, Bangs Labs, Catalog #SS05N) were washed three times with PBS, mixed with 1 mM SUVs in PBS and incubated at room temperature for 0.5-2 hours with end-over-end mixing to allow for bilayer formation. Beads were then washed three times with PBS to remove excess SUVs and incubated in 100 μl of 0.2% casein (Sigma, catalog #C5890) in PBS for 15 min before protein coupling. Unless otherwise indicated, anti-biotin AlexaFluor647-IgG (Jackson ImmunoResearch Laboratories Catalog #200-602-211, Lot #137445) was added between 3 and 30 nM, always using the lowest IgG concentration that triggered engulfment. Purified CD47^{ext}-His₁₀ was added at 1 nm. Proteins were coupled to the bilayer for 1 hour at room temperature with end-over-end mixing.

Protein Density Estimation.

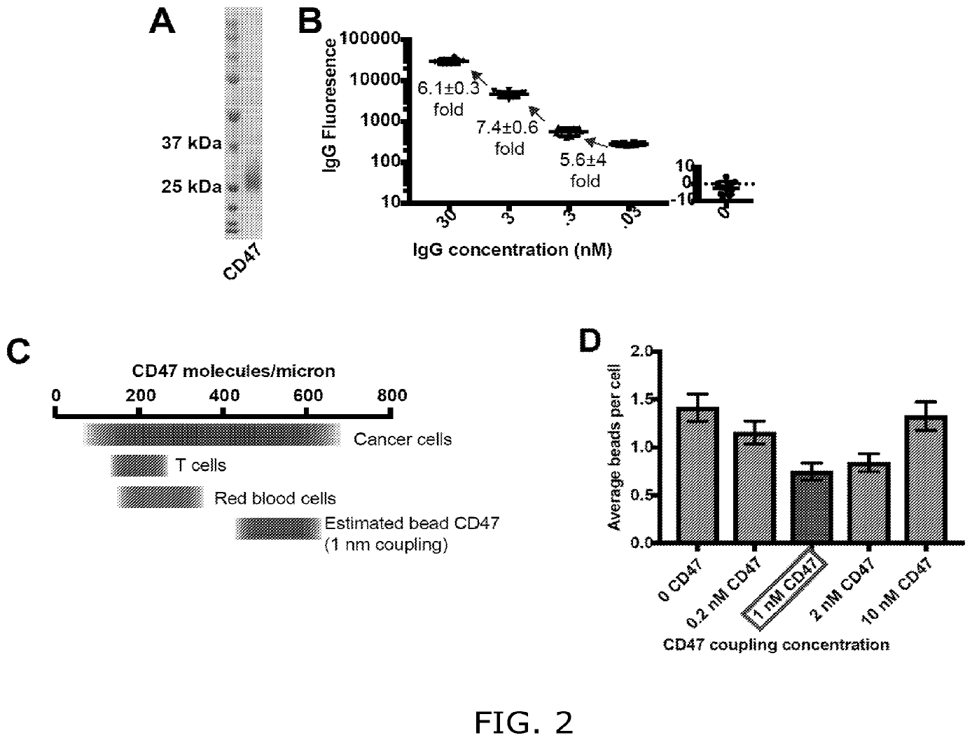

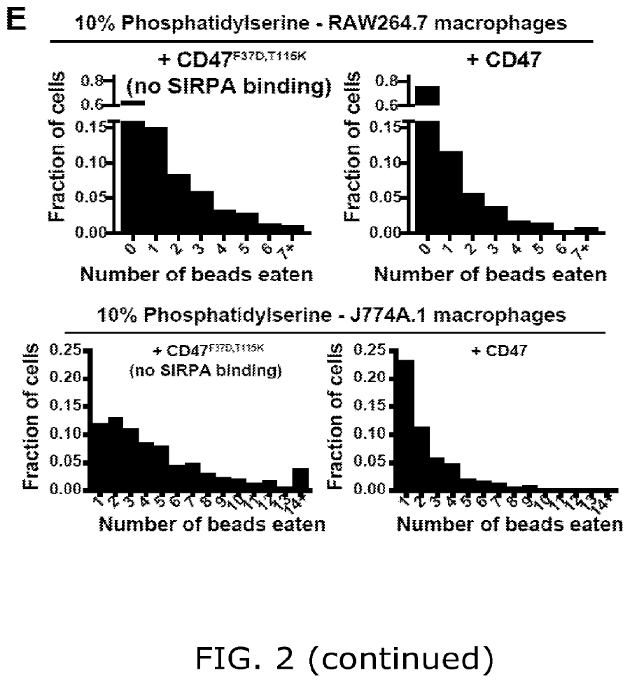

Given the high affinity of His₁₀ for Ni²⁺-DGS-NTA (0.6 nM (Hui and Vale, 2014)), and antibody-antigen interactions, it was expected that close to 100% coupling efficiency (Hui and Vale, 2014). Complete coupling would result in 600 molecules/μm² CD47 and 300 molecules/μm² IgG for the 3 nM coupling condition. This is well within the range of CD47 on the surface of a cancer cell (FIGS. 2A-2E). In addition, to estimate the amount of IgG bound to each bead, the fluorescence of IgG on the bead surface was compared to calibrated fluorescent beads (Quantum AlexaFluor 647, Bangs Lab) using confocal microscopy. Using this method, a total of 200-360 molecules/μm² of IgG was measured, which is consistent with the theoretical prediction of near complete coupling.

### Protein Purification

CD47^{ext}, His₁₀-CD47^{ext F37D, T115K}-His₁₀ (aa40-182; Uniprot Q61735) and ICAM-tagBFP-His₁₀ (O'Donoghue et al., 2013) were expressed in SF9 or HiFive cells using the Bac-to-Bac baculovirus system as described previously (Hui and Vale, 2014). Briefly, the N-terminal extracellular domain of CD47 was cloned into a modified pFastBac HT A with an upstream signal peptide from chicken RPTPσ (Chang et al., 2016). Insect cell media containing secreted proteins was harvested 72 hours after infection with baculovirus. His₁₀ proteins were purified by using Ni-NTA agarose (Qiagen, Catalog #30230), followed by size exclusion chromatography using a Superdex 200 10/300 GL column (GE Healthcare, Catalog #17517501). The purification buffer was 30 mM HEPES pH 7.4, 150 mM NaCl, 2 mM MgCl₂, 5% glycerol (CD47) or 150 mM NaCl, 50 mM HEPES pH 7.4, 5% glycerol, 2 mM TCEP (ICAM).

### Phosphopaxilin Staining

Macrophages were fixed in 4% PFA for 15 min, then permeabilized and blocked with 0.1% BSA in PBS with 0.5% Tween 20. The cells were incubated with the phosphopaxillin antibody at 1:50 dilution at 4° C. overnight before incubating with Alexa Fluor 555 anti-rabbit secondary (21428), Alexa Fluor 488 phalloidin (A12379).

### Integrin Block and Fab Generation

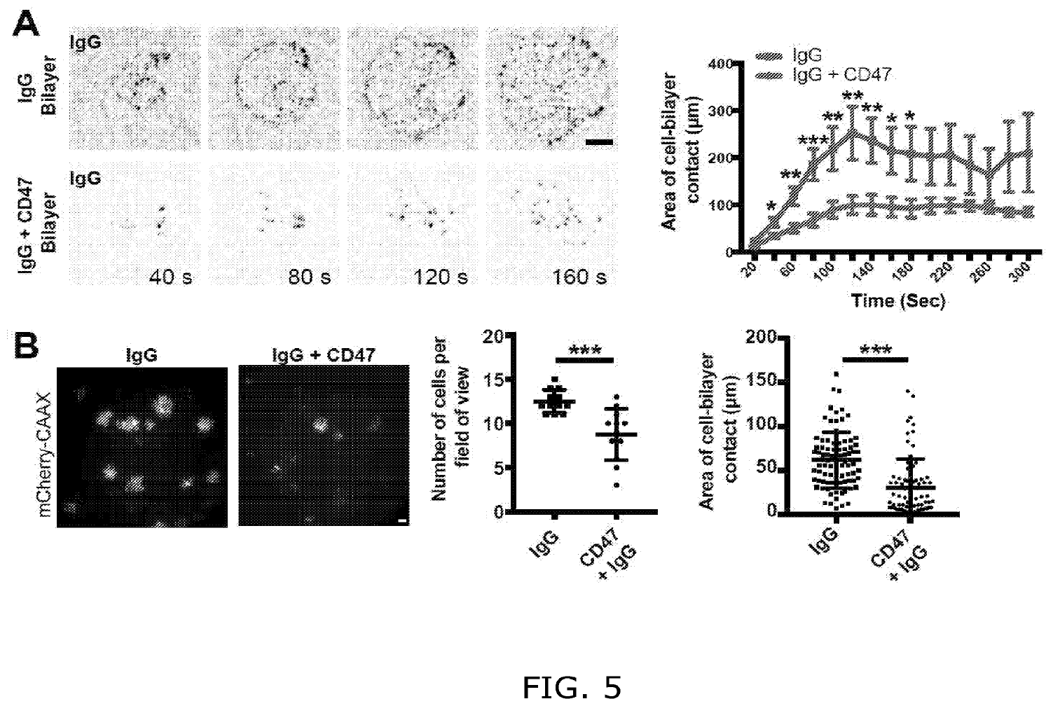

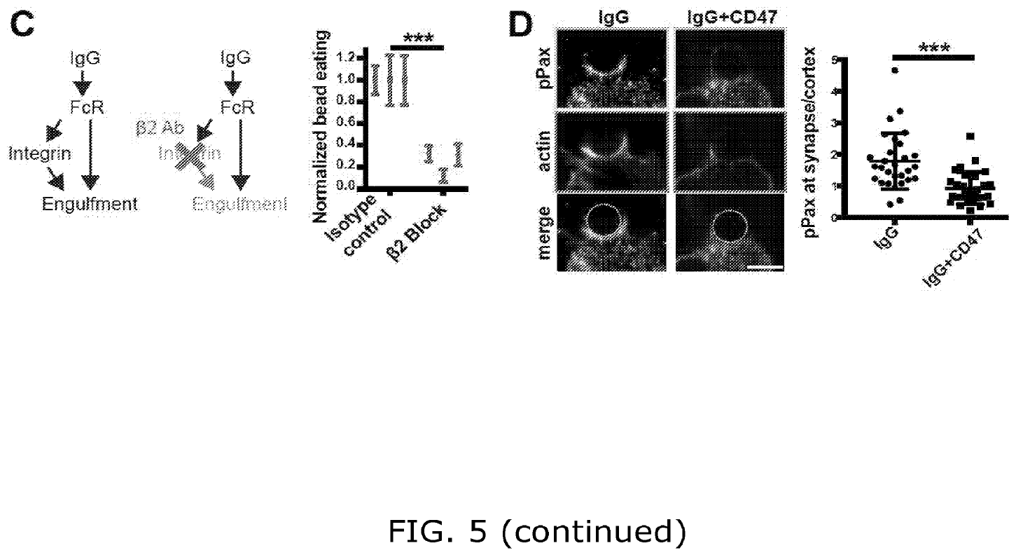

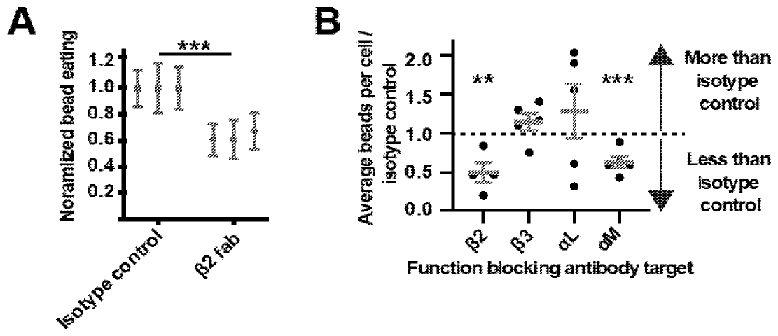

To disrupt integrin function, the blocking antibodies or isotype control indicated in the “Key Reagents” table were added to macrophages at 10 μg/ml 30 minutes before IgG-opsonized beads. To eliminate any effects of the Fe domain, we generated Fabs of the β2 blocking antibody and isotype control using the Pierce Fab separation kit (ThermoFisher 44985). In FIGS. 5A-5D and 10C, the antibodies and beads were added to macrophages in complete media containing heat inactivated serum. In FIG. 10B, macrophages were washed into serum-free HEPES imaging buffer (20 mM HEPES, 135 mM NaCl, 4 mM KCl, 10 mM glucose, 1 mM CaCl₂, 0.5 mM MgCl₂) prior to antibody treatment to eliminate any potential serum components that may serve as integrin ligands.

### Whole Cell Internalization Assay

For timelapse imaging, 30,000 primary bone marrow derived macrophages infected with GFP-CAAX were plated in a 96-well glass bottom MatriPlate (Brooks, Catalog #MGB096-1-2-LG-L). 2 hours prior to imaging, cells were washed into serum-free, phenol free DMEM for imaging. Manganese (SigmaAldrich, M8054) was added at 100 μM 30 min prior to imaging. When indicated, CD47 function-blocking antibody clone miap301 (Biolegend, 127520) was used at 10 mg/ml. 100,000 H2B-mCherry expressing L1210 cells were added and the co-culture was imaged for 8 hr.

For the end-point analysis, 30,000 primary bone marrow derived macrophages were plated in a 96-well glass bottom MatriPlate. The following morning, the macrophages were serum starved for 2 hours. Then 60,000 L1210 dyed with CFSE (ThermoFisher, C34570) were added to the well. Engulfment was allowed to proceed for 4 hr, then cells were fixed and stained with DAPI to indicate nuclei and F4/80 to label macrophages. A blinded analyzer counted the fraction of F4/80+ cells containing CFSE+DAPI+ particles in each condition.

### Confocal Imaging and Analysis

Images were acquired on a spinning disc confocal microscope (Nikon Ti-Eclipse inverted microscope with a Yokogawa spinning disk unit and an Andor iXon EM-CCD camera) equipped with a 40×0.95 NA air and a 100×1.49 NA oil immersion objective. The microscope was controlled using μManager. For TIRF imaging, images were acquired on the same microscope with a motorized TIRF arm, but using a Hamamatsu Flash 4.0 camera and the 100×1.49 NA oil immersion objective.

### Microscopy and Analysis

Images were acquired on a spinning disc confocal microscope (Nikon Ti-Eclipse inverted microscope with a Yokogawa spinning disk unit and an Andor iXon EM-CCD camera) equipped with a 40×0.95 NA air and a 100×1.49 NA oil immersion objective. The microscope was controlled using μManager (Edelstein et al., 2010). For TIRF imaging, images were acquired on the same microscope with a motorized TIRF arm, but using a Hamamatsu Flash 4.0 camera and the 100×1.49 NA oil immersion objective. Data was analyzed in ImageJ (Rueden et al., 2017; Schindelin et al., 2012).

### Quantification of Engulfment

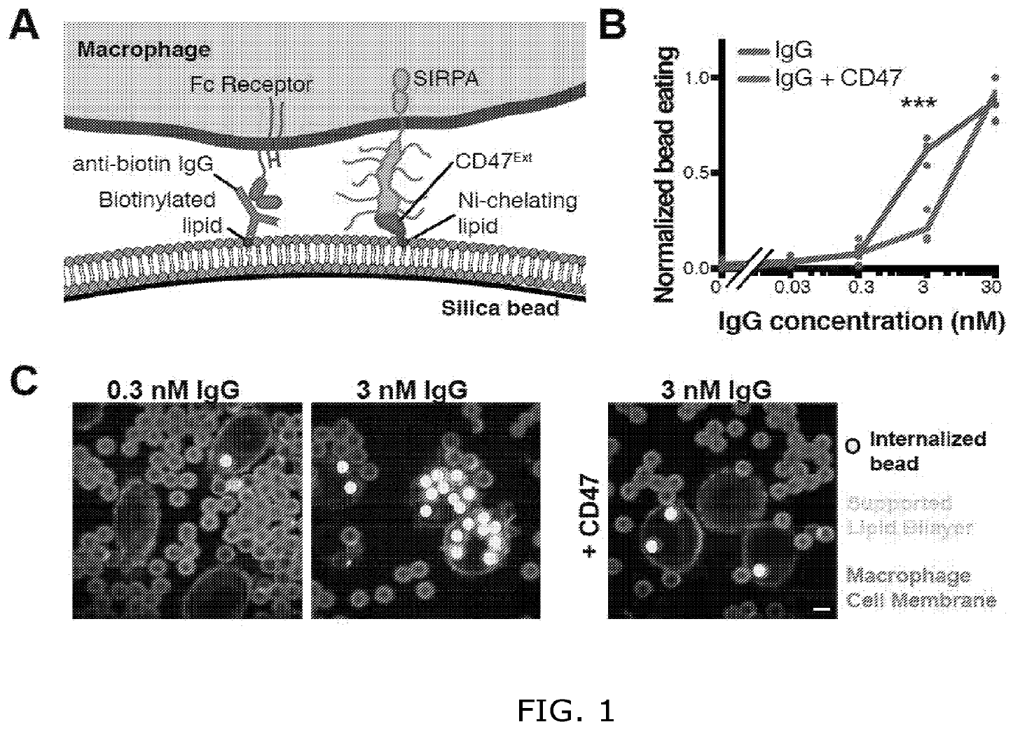

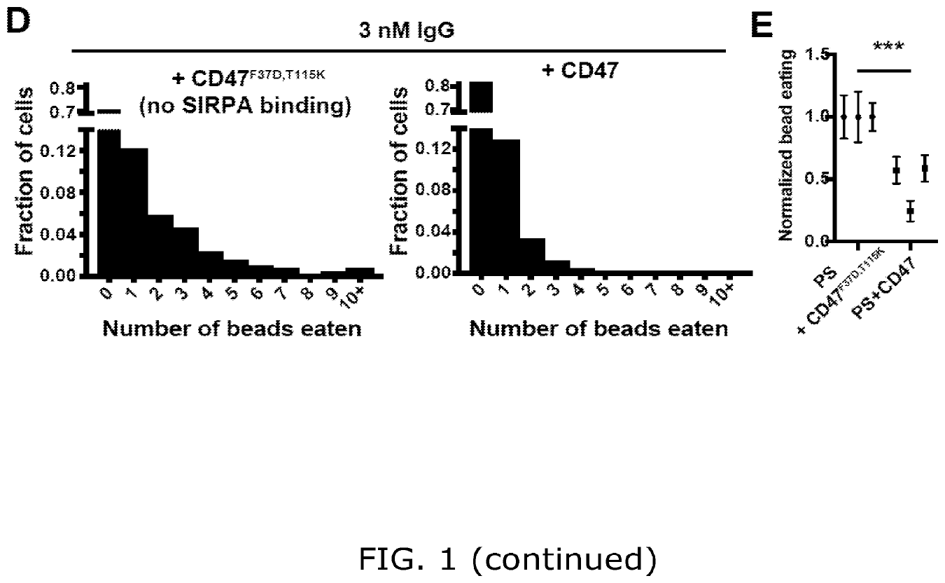

30,000 macrophages were plated in one well of a 96-well glass bottom MatriPlate (Brooks, Catalog #MGB096-1-2-LG-L) between 12 and 24 hr prior to the experiment. Unless otherwise noted, macrophages remained in culture media (DMEM with 10% heat inactivated serum) throughout the experiment. ˜8×10⁵ beads were added to well and engulfment was allowed to proceed for 30 min. Cells were fixed with 4% PFA and stained with CellMask (ThermoFisher, catalog #C10045) without membrane permeabilization to label cell boundaries. Images were acquired using the High Content Screening (HCS) Site Generator plugin in μManager (Edelstein et al., 2010). For FIGS. 1B-1D, 3G, 7F-7I, 4D, and 10A-10B, the analyzer was blinded during engulfment scoring using the position randomizer plug-in in μManager.

### TIRF Imaging

Macrophages were removed from their culture dish using 5% EDTA in PBS, two times washed and resuspended in the HEPES imaging buffer (20 mM HEPES, 135 mM NaCl, 4 mM KCl, 10 mM glucose, 1 mM CaCl₂, 0.5 mM MgCl₂) before being added to the TIRF chamber.

### Quantification of IgG Clusters and Syk Recruitment

After 15 minutes of interacting with the bilayer, cells that had spread on the bilayer surface were selected for analysis. Otsu thresholding in ImageJ was used to select IgG clusters in an unbiased manner. This selection was used to generate an ROI that was then applied to the Syk-mCherry channel. The area of the ROI (area of IgG clusters) and the mean Syk intensity within that ROI were measured.

### Pearson's Correlation Coefficient

The region of cell-bilayer contact was manually selected in ImageJ and the Pearson's correlation coefficient between AlexaFluor647-IgG and either SIRPA-GFP or Syk-mCherry for this ROI was measured using the Coloc2 plugin (Schindelin et al., 2012).

Quantification of Synapse Intensity of phosphoPaxillin, Actin and SIRPA Constructs

Phagocytic cups were selected for analysis based on the presence of clustered IgG at the cup base (SIRPA chimeras) or clear initiation of membrane extensions around the phagocytic target (actin, phosphopaxillin). The phagocytic cup and the cell cortex were traced with a line 3 pixels wide at the Z-slice with the clearest cross section of the cup. The average background intensity was measured in an adjacent region and subtracted from each measurement.

### DNA Tether Experiments

For the DNA adhesion experiments, bilayers were assembled on silica beads, blocked with 0.2% casein, and coupled to IgG at a 3 nM concentration as described above. After 15 min of IgG coupling, 1 μg/ml neutravidin was added for 20 minutes. After washing out the neutravidin, 100 nM DNA ligand strand (5′ biotin, see Key Reagents table) and 1 nM CD47ext-His10 were added and coupled for 45 min. During this time, macrophages plated in a 96 well imaging plate expressing a DNA chimeric adhesion receptor (extracellular SNAP—CD86 transmembrane domain—intracellular EGFP) were incubated with 1 uM receptor DNA strand (5′ benzylyguanine, see Key Reagents table, Farlow et al., 2013) in 100 ul buffer for 10 minutes. Cells and beads were both washed 4 times. ˜8×10⁵ beads were added to well and engulfment was allowed to proceed for 30 min in HEPES imaging buffer. The DNA ligand and receptor strand sequences along with their modifications can be found in the key resources table.

### Quantification of the Cell-Bilayer Contact Area

For FIG. 5A, time-lapse images of macrophages interacting with an IgG or IgG⁺CD47 bilayer were acquired using TIRF microscopy. The area of the cell contacting the bilayer was traced in ImageJ beginning with the first frame where the cell can be detected. Only cells with mobile IgG clusters were included. For FIG. 5B, the number of macrophage-bilayer contacts and the area was quantified in still images of live cells between 10 and 15 min after cells were added to the bilayer. All cells were included.

### Mouse Red Blood Cell Internalization Assay

For the mouse red blood cell internalization assay, 30,000 RAW264.7 macrophages (ATCC) were plated in one well of a 96-well glass bottom MatriPlate (Brooks, MGB096-1-2-LG-L) between 12 and 24 hours prior to the experiment. Mouse red blood cells (RBCs) (Innovative Research, 88R-M001) were washed into PBS and stained with CSFE (Thermo Fisher, C34554) or Alexa Fluor 488 NHS Ester (ThermoFisher, A20000) for 1 hr at room temperature. RBCs were then opsonized with C3bi as previously described (Chow et al., 2004). Briefly, RBCs were incubated with anti-mouse IgM (MyBioSource, MBS524107) for 1 hour at 37° C. A portion of RBCs were separated for IgM controls, and the remaining RBCs were incubated with C5 deficient serum (Sigma-Aldrich, C1163) for 1 hour at 37° C. Macrophages were washed into serum-free HEPES imaging buffer and incubated with 150 ng/mL PMA and 1 mM Manganese or water. ˜1×10⁶ RBCs were added to each well and engulfment was allowed to proceed for 1 hour in incubator. Cells were washed with PBS, unengulfed RBCs were lysed with water for 2 min, and cells were fixed with 4% PFA. Remaining non-lysed, non-engulfed RBCs were stained with APC anti-iC3b antibody (Biolegend, 1:200 dilution, 846106) for 30 minutes. Macrophages were stained with CellMask orange (ThermoFisher, 1:5,000 dilution) and Hoechst (Invitrogen, H3570, 1:1,000 dilution) for 15 minutes. Images were acquired using the High Content Screening (HCS) Site Generator plugin in μManager (Edelstein et al., 2010). The analyzer was blinded during engulfment scoring using the position randomizer plug-in in μManager.

### Quantification and Statistical Analysis

Statistical analysis was performed in Prism 8 (GraphPad, Inc). The statistical test used is indicated in the relevant figure legend. Sample sizes were predetermined and indicated in the relevant figure legend. In general the analyzer was blinded during analysis using either manual renaming of the files or the data randomizer plugin in μManager. The details of each quantification method and blinding strategy are included in the Methods section.

### Example 2

### CD47 Suppresses IgG and Phosphatidylserine “Eat Me” Signals

This Example describes the results of experiments performed to demonstrate that CD47-SIRPA signaling suppresses IgG and phosphatidylserine “Eat Me” signals. To study the mechanism of “Eat Me” and “Don't Eat Me” signal integration during engulfment, a reconstituted engulfment target as shown in FIG. 1A was developed and utilized. Silica beads were coated in a supported lipid bilayer to mimic the surface of a cancer cell. To activate engulfment, IgG, a well-defined “Eat Me” signal that synergizes with CD47 blockade was introduced to promote cancer cell clearance. IgG is recognized by the Fc 7 Receptor family (FcR), which activates downstream signaling and engulfment (Freeman and Grinstein, 2014). To activate SIRPA, the CD47 extracellular domain was incorporated at a surface density selected to mimic the CD47 density on cancer cells (˜600 molecules/μm², FIGS. 2A-2E).

Using this system, the effect of CD47 on engulfment was tested across a titration of IgG densities (FIGS. 2A-2E). Beads with the macrophage-like cell line RAW264.7 were mixed and measured the number of internalized beads by confocal microscopy. It was found that CD47 suppressed engulfment at intermediate IgG densities, but did not appreciably affect engulfment of targets with high densities of bound IgG (FIGS. 1B-1D). The presence of CD47 did not completely eliminate phagocytosis, but rather caused a quantitative decrease in the fraction of cells initiating engulfment and the number of beads engulfed per cell (FIGS. 1B-1C, and 2A-2E). This suppression was dependent on CD47 binding as a mutated CD47 extracellular domain that is unable to bind to SIRPA (F37D, T115K) was also unable to suppress engulfment.

Additional experiments were performed to examine whether CD47-SIRPA signaling could suppress engulfment of targets mimicking apoptotic corpses. A critical “Eat Me” signal from apoptotic corpses is phosphatidylserine, which becomes exposed on the outer leaflet of the plasma membrane during cell stress, apoptosis (Fadok et al., 1992; Poon et al., 2014), and on some cancer cells (Birge et al., 2016; Utsugi et al., 1991). It was found that engulfment of beads containing 10% phosphatidylserine in the supported lipid bilayer was inhibited by the inclusion of CD47 on the bilayer (see, e.g., FIGS. 1E and 2A-2E). Together, these data show that CD47-SIRPA signaling can block engulfment driven by IgG and phosphatidylserine. Moreover, bilayer-coated beads provide a well-defined and tunable platform for studying the integration of “Eat Me” and “Don't Eat Me” signals during engulfment.

### Example 3

### CD47 Ligation Relocalizes SIRPA to the Phagocytic Synapse

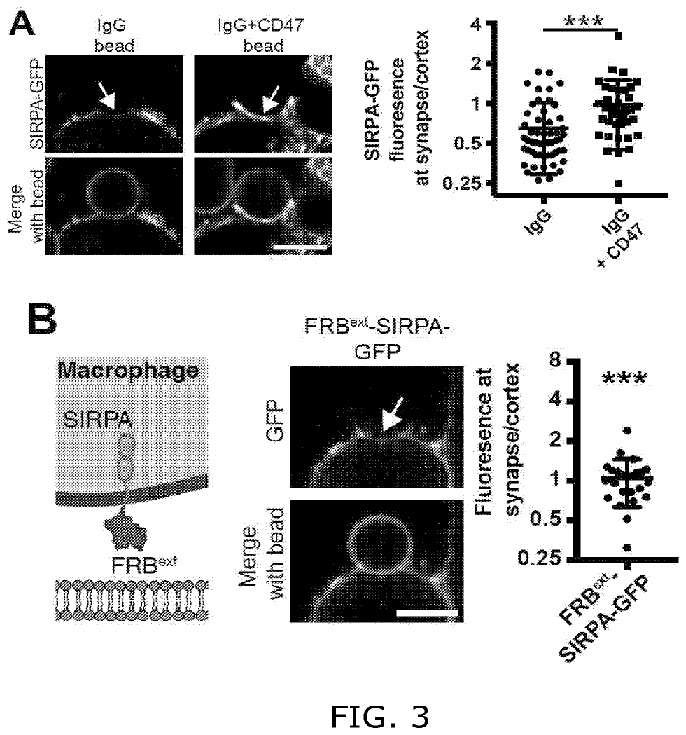

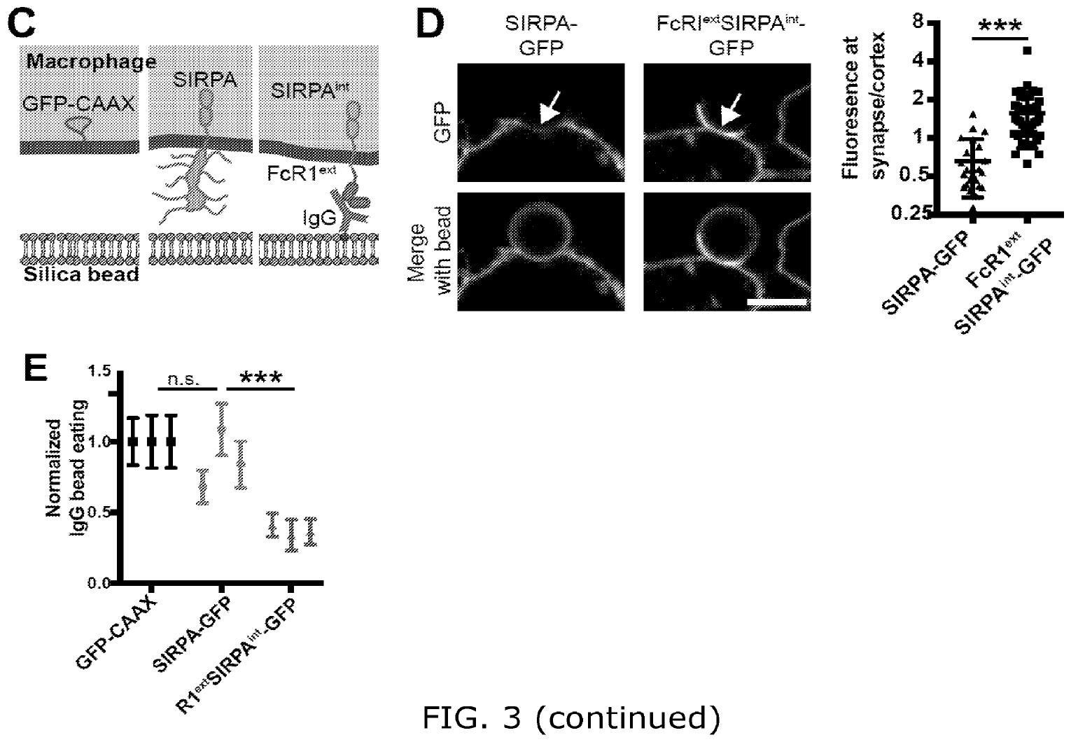

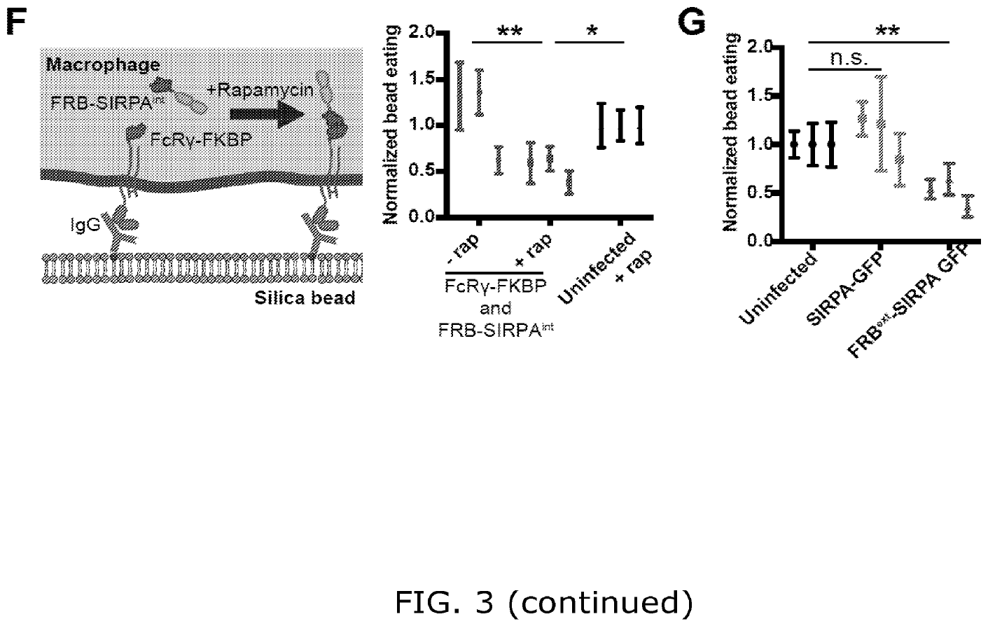

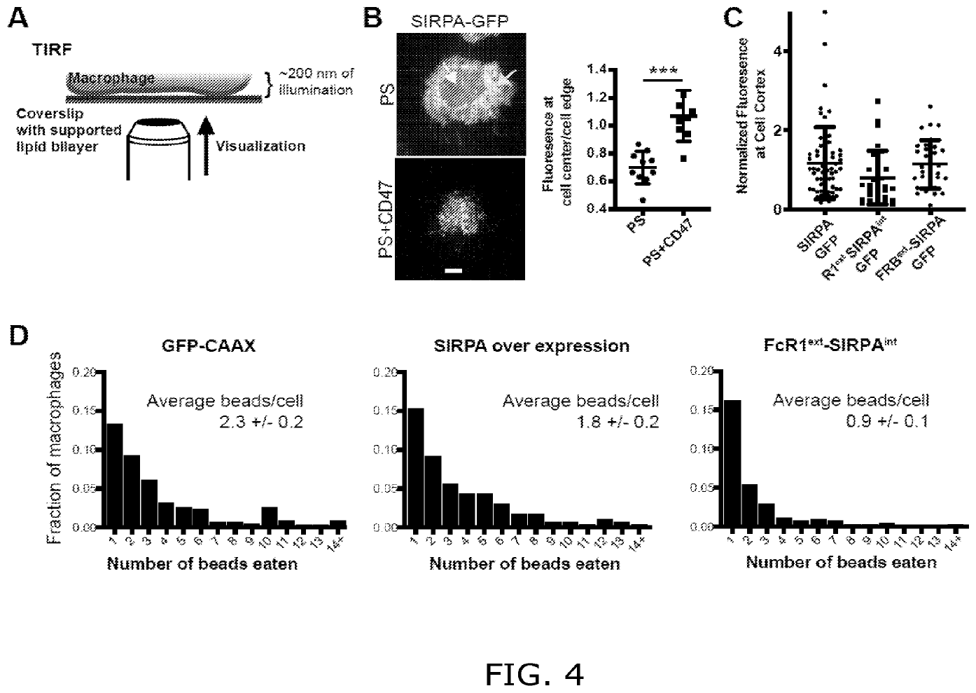

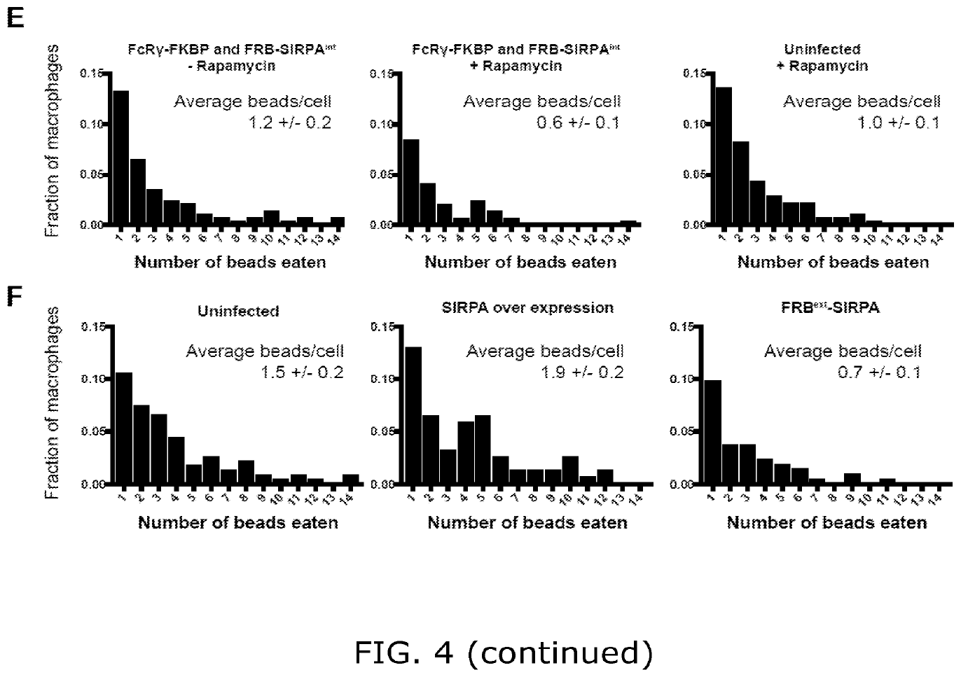

This Example describes the results of experiments performed to demonstrate that CD47 ligation relocalizes SIRPA to the phagocytic synapse. These experiments were performed to determine the mechanism by which CD47 ligation regulates SIRPA activity. SIRPA localization during phagocytosis of IgG-coated beads was first examined. When not bound to CD47, SIRPA was segregated away from the phagocytic cup that enveloped IgG-coated beads (FIG. 3A). Similarly, SIRPA was depleted at the center of the immunological synapse between a macrophage and a supported lipid bilayer containing phosphatidylserine (FIGS. 4A-4F). In contrast, in the presence of CD47, SIRPA remained at the phagocytic cup (FIG. 3A). These data demonstrate that unligated SIRPA is excluded from the phagocytic synapse, whereas CD47-bound SIRPA remains at the phagocytic synapse.

Additional experiments were performed to address the mechanism of SIRPA segregation away from the phagocytic cup in the presence of IgG and absence of CD47. It was hypothesized that exclusion of unligated SIRPA from the synapse could be driven by its heavily glycosylated extracellular domain, either by interactions with the surrounding glycocalyx or steric exclusion from the spatially restricted phagocytic synapse. A SIRPA chimeric receptor was therefore created where the extracellular domain was replaced with a small, inert protein domain (FRB^{ext}-SIRPA; FIG. 3B). Unlike full length SIRPA, FRB^{ext}-SIRPA was not segregated away from the cell-target synapse (FIG. 3C). This result demonstrates that the extracellular domain of SIRPA is required for SIRPA exclusion from the phagocytic cup.

Exclusion of bulky phosphatases, like CD45, is driven by the steric constraints of a small distance between the macrophage and target membrane. It was then hypothesized that the short membrane-membrane distance driven by FcR-IgG ligation (˜11.5 nm) may be sufficient to exclude SIRPA. To test this hypothesis, a series of synthetic tethers varying in length was generated (FIG. 3D). In the macrophage, synthetic transmembrane proteins containing an intracellular GFP were expressed, and an extracellular domain with 0, 1, 3 or 5 repeats of a synthetic FNIII protein, Fibcon (Bakalar et al., 2018; Jacobs et al., 2012), plus half of an inducible dimerization system (Fib0FRB to Fib5FRB). The other half of the inducible dimerization system was attached to bilayer-coated beads (FKBP-His10). Beads were then tethered to the macrophages in the absence of IgG or CD47 by adding a rapamycin analog to induce dimerization between the synthetic proteins (Spencer et al., 1993). SIRPA exclusion from the phagocytic synapse was quantified. It was found that the tethers containing no Fibcon repeats (FRB-FKBP alone, ˜6 nm) or one Fibcon repeat (Fib1FRB-FKBP, ˜9.5 nm) drove SIRPA exclusion of a similar magnitude to FcR-IgG ligation (FIGS. 2D and E). The efficiency of SIRPA exclusion decreased with longer tether lengths (Fib3FRB-FKBP, 16.5 nm when fully extended; and Fib5FRB-FKBP, 21.5 nm). Together, these data indicate that SIRPA exclusion can be controlled by altering the height of the immunological synapse.

### Example 4

### Targeting SIRPA to the Phagocytic Synapse Suppresses Engulfment

This Example describes the results of experiments performed to demonstrate that targeting SIRPA to the phagocytic synapse suppresses engulfment. Receptor activation by Src family kinases at the phagocytic cup is favored due to exclusion of bulky phosphatases like CD45 (Freeman et al., 2016; Goodridge et al., 2011). SIRPA contains two immune tyrosine inhibitory motifs (ITIMs) that are phosphorylated by Src family kinases and essential for downstream signaling. It was hypothesized that positioning SIRPA at the phagocytic cup may drive ITIM phosphorylation and receptor activation. To distinguish between the effects of CD47 binding and synapse localization, a chimeric SIRPA receptor, which localized to the phagocytic synapse in the absence of CD47, was developed. The SIRPA extracellular domain was replaced with the IgG-binding extracellular domain of the FcγRI α chain (FIG. 3C; termed FcR1^{ext}-SIRPA^{int}). This receptor is driven into the synapse by IgG binding instead of CD47 (FIG. 3D). Expression of this synapse-localized chimera suppressed engulfment of IgG-coated beads in the absence of CD47 (FIGS. 3E, 4A-4F). As a control, a chimeric construct with the four tyrosines of the ITIM domains mutated to phenylalanines, prohibiting phosphorylation and activation of SIRPA (FcR3^{ext}-SIRPA 4F^{int}) was expressed. This construct did not affect engulfment, suggesting that the inhibitory effect of the SIRPA chimera is signaling dependent (FIG. 5B). Thus, targeting SIRPA to the phagocytic cup is sufficient to inhibit engulfment, even in the absence of its natural ligand CD47.

As an alternative strategy to control the localization of SIRPA activity, additional experiments were performed by using a chemically inducible dimerization system (Spencer et al., 1993). One half of the chemically inducible dimer was fused to FcR (FcR γ chain-FKBP) and the second to a soluble SIRPA intracellular domain (FRB-SIRPA^{int}, FIG. 3F). In the presence of the small molecule rapamycin, FKBP and FRB form a high-affinity dimer (Spencer et al., 1993), thereby recruiting the SIRPA intracellular domain to FcR. In the absence of rapamycin, cells efficiently engulfed IgG-coated beads (FIGS. 3F, 4A-4F). In contrast, rapamycin-induced recruitment of the SIRPA intracellular domain to the FcR γ chain significantly suppressed engulfment (FIG. 3F). When the ITIM domain of SIRPA was mutated, this construct no longer affected engulfment (FIG. 5C).

Next, additional experiments were performed to examine the extracellular domain truncation of SIRPA (FRB^{ext}-SIRPA), which was not excluded from the phagocytic synapse (FIGS. 3B-3C) to determine if eliminating SIRPA exclusion is sufficient to suppress engulfment. FRB^{ext}-SIRPA constitutively suppressed engulfment (FIG. 3G), demonstrating that exclusion of SIRPA is essential for efficient engulfment of targets presenting “Eat Me” signals. Taken together, these experiments show that exclusion of unligated SIRPA is essential for efficient phagocytosis and that CD47 activates SIRPA by positioning SIRPA at the phagocytic synapse.

### Example 5

### FcR Phosphorylation is not a Major Target of CD47-SIRPA Signaling

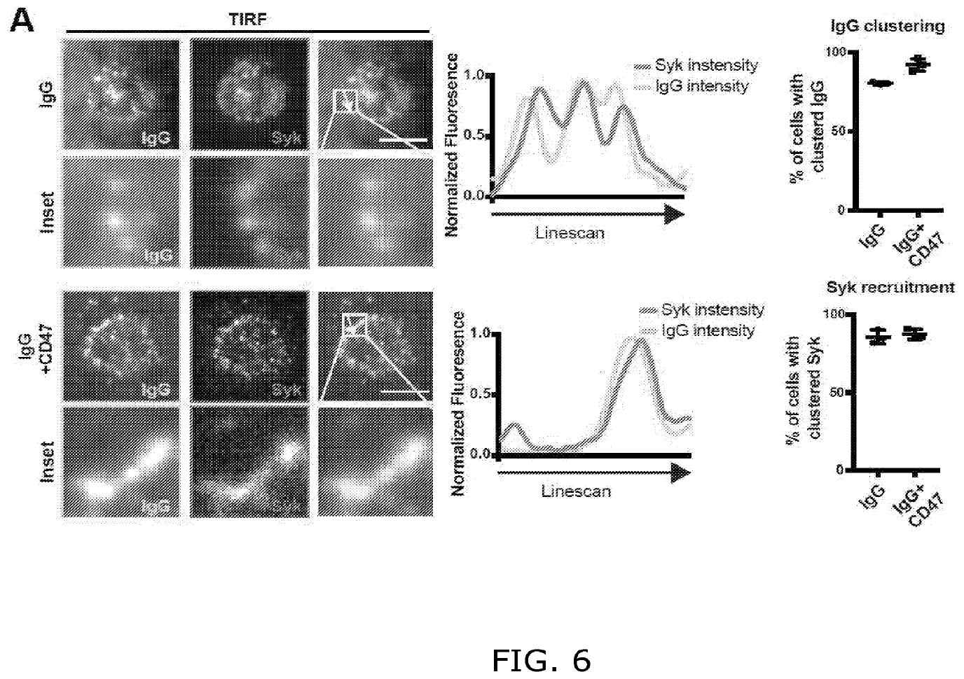

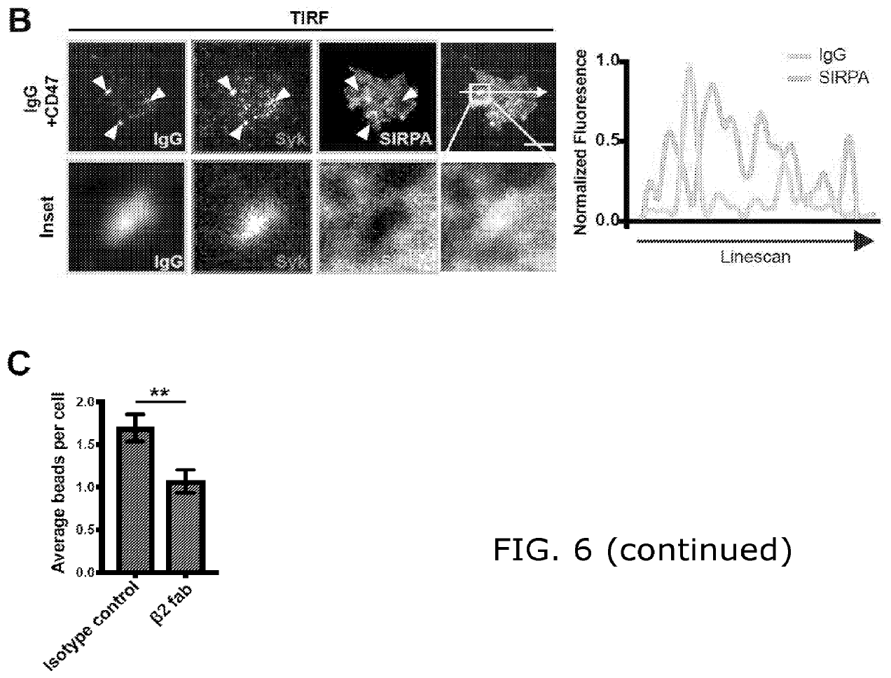

This Example describes the results of experiments performed to demonstrate that CD47 does not suppress engulfment by altering Syk recruitment to IgG microclusters and that FcR phosphorylation is not a major target of CD47-SIRPA signaling. These experiments were performed to determine how activated SIRPA inhibits engulfment. Phosphorylated SIRPA recruits the phosphatases SHP-1 and SHP-2 via their phosphobinding SH2 domains but the downstream targets of SHP-1 and SHP-2 are not known (Fujioka et al., 1996; Noguchi et al., 1996; Okazawa et al., 2005; Oldenborg et al., 2001; Veillette et al., 1998). One potential target of SIRPA-bound SHP phosphatases is FcR itself. TIRF microscopy was used to examine the initial steps in the engulfment signaling cascade with high temporal and spatial resolution. When macrophages interacted with an IgG-bound supported lipid bilayer, the cells formed IgG microclusters that recruited Syk (FIGS. 5A, 6A-6C, and Movie S1 (Lin et al., 2016) (still images from Movie S1 are shown in FIG. 5A). When CD47 was present, these microclusters still formed and recruited Syk (FIGS. 5A, 6A-6C, and Movie S2) (still images from Movie S2 are shown in FIG. 5A). When static images of macrophages that had landed on a bilayer containing IgG and CD47, or IgG and the inactive CD47^{F37D, T115K} were compared, no significant difference in the fraction of cells forming IgG microclusters or the total area of the IgG microclusters under the cells was detected. There was also no significant difference in the fraction of cells containing Syk microclusters or the amount of Syk-mCherry recruited to these clusters (FIGS. 6B-6C). Further, when SIRPA localization at the cell-target interface at high resolution was examined, it was observed that, even in the presence of CD47, SIRPA did not co-localize with FcR clusters when macrophages landed on a bilayer containing IgG and CD47, indicating that SIRPA is not positioned to dampen receptor activation (FIGS. 6A-6C). Overall, this suggests that changes to FcR activation and Syk recruitment are unlikely to account for the effect of SIRPA, consistent with previous biochemical observations (Okazawa et al., 2005; Tsai and Discher, 2008).

### Example 6

### CD47 Prevents Integrin Activation

This Example describes the results of experiments performed to assess the dynamics of cells landing on functionalized supported lipid bilayers. During the TIRF experiments described herein, it was observed that cells on IgG-coated bilayers spread across the bilayer surface (FIG. 5A, Movie S1) (still images from Movie S1 are shown in FIG. 5A). In contrast, macrophages encountering an IgG and CD47-containing bilayer exhibited reduced cell spreading (FIGS. 5A and 5B, Movie S2) (still images from Movie S2 are shown in FIG. 5A). TIRF imaging at a static timepoint revealed that fewer macrophages were interacting with the bilayer, and those interacting had a smaller footprint (FIG. 5B). These data show that CD47 inhibits cell spreading across a target substrate.

Cell spreading is thought to involve activation of integrins and the actin cytoskeleton (Springer and Dustin, 2011). Inactive integrins exist in a low affinity, bent confirmation (Springer and Dustin, 2011). Upon activation, the extracellular domain extends into an open conformation that can bind many ligands with high affinity (Freeman and Grinstein, 2014; Springer and Dustin, 2011). FcR activation stimulates inside-out activation of integrins (Dupuy and Caron, 2008; Jones et al., 1998). Activated integrins can then promote engulfment, either by increasing adhesion to the target particle or by providing a platform for intracellular signaling and actin assembly (Dupuy and Caron, 2008; Wong et al., 2016). It was found that inhibiting integrin with a P2 integrin function-blocking antibody (2E6) or Fab dramatically decreased the efficiency of IgG-mediated engulfment (FIGS. 5C, 6A-6C, and 10A-10B). It was also possible to detect a role for αM integrin in engulfment, but not for β3 or αL (FIGS. 10A-10B). Thus, blocking αMβ2 integrin is sufficient to suppress engulfment.

Because integrin is required for cell spreading and engulfment (Springer and Dustin, 2011), it was hypothesized that CD47-SIRPA signaling may inhibit engulfment by preventing inside-out activation of integrin. To determine if integrin activation was inhibited by CD47, additional experiments were performed to examine the localization of phosphopaxillin, which is specifically recruited to sites of integrin activation (Geiger et al., 2009). It was found that the enrichment of phospho-paxillin at the interface of the macrophage with an IgG-coated bead was substantially diminished by the simultaneous presence of CD47 on the bead (FIG. 5D). Together, these data indicate that CD47-SIRPA signaling prevents integrin activation.

### Example 7

### Activating Integrin Bypasses CD47-SIRPA Inhibitory Signaling

This Example describes the results of experiments performed to demonstrate that activation of integrin bypasses CD47-SIRPA inhibitory signaling. CD47-SIRPA has previously been reported to affect phosphorylation of multiple proteins, including paxillin and myosin. Experimental data presented herein also demonstrates that SIRPA inhibited F-actin accumulation at the phagocytic cup (FIG. 5D). It is not clear which of these pathways is a direct target of CD47 signaling and which is a secondary effect of altered upstream signaling. It was hypothesized that if SIRPA signaling suppresses engulfment primarily by inhibiting integrin inside-out activation, then directly activating integrin might bypass SIRPA-mediated inhibition and permit bead engulfment (FIGS. 7A and 9A). Alternatively, if the target of CD47-SIRPA signaling is in a parallel pathway or downstream of integrin activation, then activating integrin should not rescue engulfment following SIRPA activation. To activate integrin, macrophages were treated with manganese, which locks integrin into a high-affinity open conformation (Dransfield et al., 1992). It was found that macrophages treated with 1 mM manganese engulfed beads with a similar efficiency whether or not CD47 was conjugated to the supported lipid bilayer (FIGS. 7B and 9B). Importantly, manganese did not trigger bead engulfment on its own or dramatically enhance engulfment of IgG-coated beads in the absence of CD47 (FIGS. 7B-7C and 9B-9C), establishing that increasing integrin activation is not sufficient to trigger engulfment. Thus, a manganese-induced increase in engulfment was specific to beads coated with CD47 and IgG.

As an alternative strategy to activate integrins, macrophages were incubated with beads containing a surplus of high affinity integrin ligand, ICAM-1 (Springer and Dustin, 2011). ICAM-1 was sufficient to activate integrin and recruit phophopaxillin even in the presence of CD47 (FIGS. 5D, 7D and 9D). Inclusion of high concentrations of ICAM-1 abrogated the inhibitory effect of CD47 on phagocytosis, but did not dramatically alter the engulfment efficiency of IgG coated beads in the absence of CD47 (e.g., FIGS. 7E and 9E). Despite the presence of CD47, ICAM-1-bound beads had similar levels of actin accumulation as beads lacking CD47 (e.g., FIGS. 10D and 5D). This demonstrates that activating integrins restores the ability of a macrophage to engulf targets in the presence of CD47. Together, these data suggest that inside-out activation of integrins may be a primary target for repression following CD47-SIRPA engagement.

CD47 has previously been reported to suppress complement-mediated phagocytosis. Because complement directly activates αMβ2 integrin, additional experiments were performed to determine if preventing integrin activation could account for the suppressive effect of CD47 in complement-mediated phagocytosis. To address this, experiments were performed to examine whether manganese treatment could increase macrophage engulfment of complement-opsonized mouse red blood cells (RBCs), which present CD47 on their surface (FIGS. 7F and 9F). It was found that activating integrin with 1 mM manganese dramatically increased engulfment of complement-opsonized RBCs but not control IgM-treated RBCs. This demonstrates that activating integrin enhances complement mediated engulfment, and is consistent with integrin activation bypassing the suppressive CD47 signal on complement-opsonized red blood cells.

### Example 8

### Adhesion Alone is not Sufficient Nor Bypass CD47

Additional experiments were performed to clarify whether integrin bypassed the CD47 signal by acting simply as a physical tether, or if intracellular integrin signaling was required. To distinguish between a tethering role and a signaling role for integrins, a DNA-based synthetic receptor was created that tethered the bilayer-coated beads to the macrophage but contained no intracellular signaling domains (FIG. 9G). It was observed that unlike integrin activation, this inert tether was unable to bypass the suppressive CD47 signal (FIG. 9H). This result indicates that adhesion alone cannot overcome the effect of CD47 and that the intracellular signaling capabilities of integrin are essential.

### Example 9

### Integrin Activation Drives Cancer Cell Engulfment

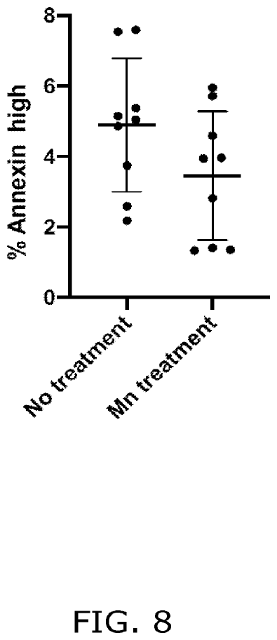

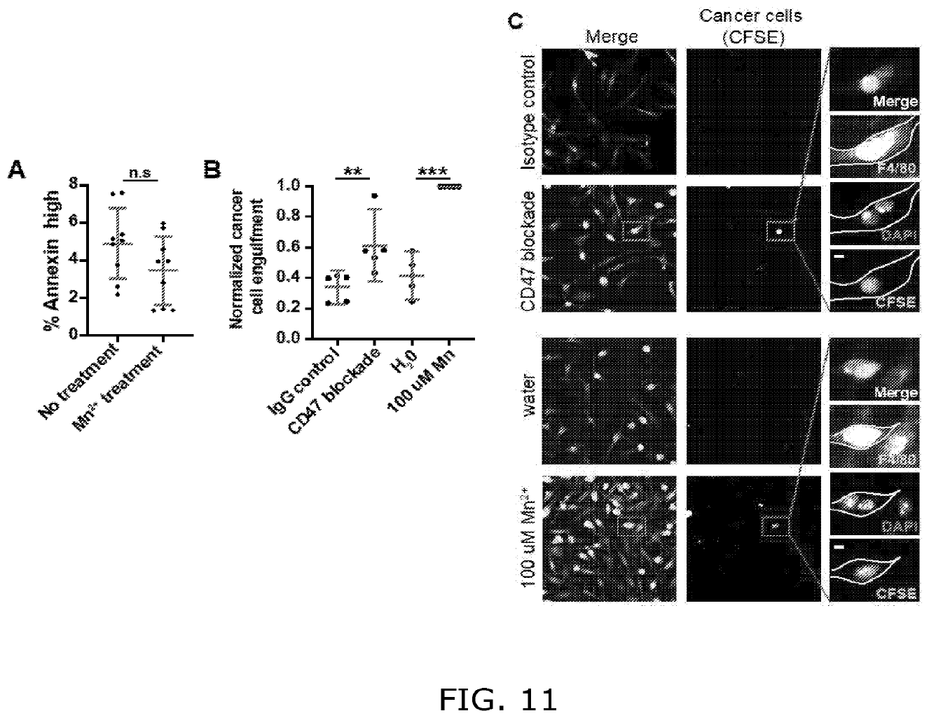

This Example describes the results of experiments performed to demonstrate that activation of integrins drives cancer cell engulfment. Many cancer cells overexpress CD47 to evade the innate immune system despite increased expression of “Eat Me” signals such as calreticulin or phosphatidylserine (Birge et al., 2016; Chao et al., 2010b; Gardai et al., 2005; Utsugi et al., 1991). Blocking CD47 with a therapeutic antibody allows “Eat Me” signals to dominate, resulting in engulfment of whole cancer cells (Jaiswal et al., 2009; Majeti et al., 2009). It was hypothesized that exogenous activation of integrin would bypass the CD47 signal on the surface of cancer cells, allowing for engulfment. To test this, bone marrow derived mouse macrophages expressing a membrane tethered GFP (GFP-CAAX) were incubated with a CD47-positive murine leukemia line, L1210, expressing nuclear H2B-mCherry (Chen et al., 2017). Macrophage-cancer cell interactions were then imaged for 8 hours. It was found that activating integrins with 100 μM manganese increased the ability of macrophages to engulf cancer cells, reaching a similar efficiency as treatment with a CD47 function-blocking antibody (FIG. 7G-7H; Movie S3). These images correspond to frames taken from a movie (Movie S3) showing a macrophage (green) engulfs a cancer cell (magenta). The macrophage was a mouse bone marrow derived macrophage expressing GFP-caax labeling the cell membrane. The cancer cells was a mouse leukemia cell line L1210 expressing H2B-mCherry which labeled the nucleus of the cancer cell. It was found that the cancer cell was engulfed and degraded, as can be observed by the release of mCherry from the nucleus. Manganese did not directly affect cancer cell viability over the time course of this experiment (FIGS. 8 and 11A-11C). To confirm this result, L1210 cancer cells were dyed with CFSE. These dyed cancer cells were then incubated with primary bone marrow derived macrophages for 2 hours at a 2:1 cancer cell:macrophage ratio. It was observed that manganese increased whole cell engulfment in this assay as well (FIGS. 11A-11C). These data indicate that activating integrins bypasses the suppressive CD47 signal on the surface of cancer cells.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

TABLE 1
Techniques, strategies, and reagents.

| | | |
|---|---|---|
| REAGENT or RESOURCE | SOURCE | IDENTIFIER |

Antibodies

| | | |
|---|---|---|
| anti-pPaxillin | Cell Signaling, Tech. | Cat#2541 |
| AlexaFluor 647 anti-biotin IgG | Jackson Immuno Labs | Cat#200-602-211 |
| CD 18 monoclonal antibody 2E6 | ThermoFisher | Cat# MA 1805 |
| Armenian hamster isotype | ThermoFisher | Cat#16-4888-81 |
| control (for 2E6) | | |

Chemicals, Peptides, and Recombinant Proteins

| | | |
|---|---|---|
| Alexa Fluor 488 Phalloidin | Thermo/Molecular | Cat# A12379 |
| | Probes | |
| Manganese | Sigma Aldrich | Cat# M8054 |
| POPS | Avanti | Cat# 840035 |
| Biotinyl Cap PE | Avanti | Cat# 870273 |
| POPC | Avanti | Cat# 850457 |
| Ni2+-DGS-NTA | Avanti | Cat# 790404 |
| PEG5000-PE | Avanti | Cat# 880230 |
| atto390 DOPE | ATTO-TEC GmbH | Cat# AD 390-161 |
| M-CSF | Peprotech | Cat# 315-02 |

Critical Commercial Assays

| | | |
|---|---|---|
| Pierce Fab Preparation Kit | ThermoFisher | Cat# 44985 |

Experimental Models: Cell Lines

| | | |
|---|---|---|
| HEK293T cells | UCSF Cell Culture | |
| | Facility | |
| Hi Fives | ThermoFisher | Cat# BTI-TN-5B1-4 |
| Sf9 | ThermoFisher | Cat# 11496015 |
| Raw264.7 Macrophages | ATCC | Cat# ATCC ® TIB-71 ™ |
| J774A.1 Macrophages | UCSF Cell Culture | |
| | Facility | |

Experimental Models: Organisms/Strains

| | |
|---|---|
| C57BL/6J | Bone marrow-derived |
| | macrophages generated |
| | as described: PMID: |
| | 21356739 |

Recombinant DNA

| | | |
|---|---|---|
| SIRPA-GFP | this paper | CDS: aa1-513 UniProtKB - P97797 |
| | | (SHPS1_MOUSE), Linker: ADPPVAT, |
| | | Fluorophore: mGFP |
| Syk-mCherry | this paper | CDS: aa1-583 UniProtKB - P48025 |
| | | (KSYK_MOUSE), Linker: ADPPVAT, |
| | | Fluorophore: mCherry |
| SIRPA^{int}_Fcr1^{Ext} | this paper | CDS: Extracellular: aa1-297 UniProtKB |
| | | P26151 (FCGR1_MOUSE) Linker: |
| | | GSGSGSGGGGSS, TM and intracellular: |
| | | aa374-513, UniProtKB - P97797 |
| | | (SHPS1_MOUSE), Linker: ADPPVAT, |
| | | Fluorophore: mGFP |
| FRB^{ext}_SIRPA | this paper | Signal peptide: aa 1-20 Uniprot Q9UM88 (beta |
| | | 2-microglobulin) Extracellular: FRB from Ariad |
| | | Heterodimeriztion kit (see James and Vale, |
| | | 2012), Linker: GSGSGSGGGGSS, TM and |
| | | intracellular: aa374-513, UniProtKB - P97797 |
| | | (SHPS1_MOUSE), Linker: ADPPVAT, |
| | | Fluorophore: mGFP |
| Fcgamma-FKBP p2a FRB- | this paper | FcR gamma chain: aa1-86 UniProtKB - |
| Sirpa^{int}_mch | | P20491 (FCERG_MOUSE) Linker: |
| | | GSGSGGGGSS, FKBP(F36V) from Ariad |
| | | Heterodimerization kit (see James and Vale |
| | | 2012) p2a; FRB(T2098L), SIRPAint: aa395- |
| | | 511 ProtKB - P97797 (SHPS1_MOUSE), |
| | | Linker: ADPPVAT, Fluorophore: mCherry |
| ICAM-tagBFP-His10 | DOI: 10.7554/eLife.00778 | |
| CD47ext-His10 | this paper | Signal peptide: from RPTPσ, CD47ext: aa40- |
| | | 182 (Uniprot Q61735), Linker: TS, Tag: His10 |
| CD47(F37D, T115K)ext-His10 | this paper | As above, except F37D, T115K |
| pMD2.G lentiviral plasmid | D. Stainier, Max Planck; | Addgene 12259 |
| | VSV-G envelope | |
| pCMV-dR8.91 | DOI: | Current Addgene 8455 |
| | 10.1038/nature11220. | |
| pHRSIN-CSGW | DOI: | |
| | 10.1038/nature11220. | |

Software and Algorithms

| | | |
|---|---|---|
| ImageJ | NIH | |
| Illustrator | Adobe | CS6 |
| Photoshop | Adobe | CS6 |
| Fiji | https://fiji.sc/ | |
| Prism | GraphPad | 8 |
| Micromanager | DOI: 10.14440/jbm.2014.36 | |

Other

| | | |
|---|---|---|
| 5 um silica microspheres | Bangs | Cat# SS05N |
| Coverslips for TIRF chambers | Ibidi | Cat# 10812 |
| MatriPlate | Brooks | Cat# MGB096-1-2-LG-L |

### REFERENCES

Advani, R., Flinn, I., Popplewell, L., Forero, A., Bartlett, N. L., Ghosh, N., Kline, J., Roschewski, M., LaCasce, A., Collins, G. P., et al. (2018). CD47 Blockade by Hu5F9-G4 and Rituximab in Non-Hodgkin's Lymphoma. N. Engl. J. Med. 379, 1711-1721.
Alenghat, F. J., Baca, Q. J., Rubin, N. T., Pao, L. I., Matozaki, T., Lowell, C. A., Golan, D. E., Neel, B. G., and Swanson, K. D. (2012). Macrophages require Skap2 and Sirpα for integrin-stimulated cytoskeletal rearrangement. J. Cell Sci. 125, 5535-5545.
Bakalar, M. H., Joffe, A. M., Schmid, E. M., Son, S., Podolski, M., and Correspondence, D. A. F. (2018). Size-Dependent Segregation Controls Macrophage Phagocytosis of Antibody-Opsonized Targets. Cell 174, 131-142.
Barclay, A. N., and Brown, M. H. (2006). The SIRP family of receptors and immune regulation. Nat. Rev. Immunol. 6, 457-464.
Birge, R. B., Boeltz, S., Kumar, S., Carlson, J., Wanderley, J., Calianese, D., Barcinski, M., Brekken, R. A., Huang, X., Hutchins, J. T., et al. (2016). Phosphatidylserine is a global immunosuppressive signal in efferocytosis, infectious disease, and cancer. Cell Death Differ. 23, 962-978.
Campbell, H., Heidema, C., Pilarczyk, D. G., and DeMali, K. A. (2018). SHP-2 is activated in response to force on E-cadherin and dephosphorylates vinculin Y822. J. Cell Sci. 131, jcs216648.
Caron, E., Self, A. J., and Hall, A. (2000). The GTPase Rap1 controls functional activation of macrophage integrin alphaMbeta2 by LPS and other inflammatory mediators. Curr. Biol. 10, 974-978.
Cham, L. B., Bear, L., Dulgeroff, T., Caspi, M., Weissman, I. L., Lang, K. S., and Correspondence, K. J. H. (2020). Immunotherapeutic Blockade of CD47 Inhibitory Signaling Enhances Innate and Adaptive Immune Responses to Viral Infection. Cell Reports 31, 107494.
Chang, V. T., Fernandes, R. A., Ganzinger, K. A., Lee, S. F., Siebold, C., McColl, J., Jonsson, P., Palayret, M., Harlos, K., Coles, C. H., et al. (2016). Initiation of T cell signaling by CD45 segregation at “close contacts”. Nat. Immunol. 17, 574-582.
Chao, M. P., Alizadeh, A. A., Tang, C., Myklebust, J. H., Varghese, B., Gill, S., Jan, M., Cha, A. C., Chan, C. K., Tan, B. T., et al. (2010a). Anti-CD47 Antibody Synergizes with Rituximab to Promote Phagocytosis and Eradicate Non-Hodgkin Lymphoma. Cell 142, 699-713.
Chao, M. P., Jaiswal, S., Weissman-Tsukamoto, R., Alizadeh, A. A., Gentles, A. J., Volkmer, J., Weiskopf, K., Willingham, S. B., Raveh, T., Park, C. Y., et al. (2010b). Calreticulin Is the Dominant Pro-Phagocytic Signal on Multiple Human Cancers and Is Counterbalanced by CD47. Sci. Transl. Med. 2, 63ra94-63ra94.
Chao, M. P., Weissman, I. L., and Majeti, R. (2012). The CD47-SIRPα pathway in cancer immune evasion and potential therapeutic implications. Curr. Opin. Immunol. 24, 225-232.
Chen, J., Zhong, M.-C., Guo, H., Davidson, D., Mishel, S., Lu, Y., Rhee, I., Pèrez-Quintero, L.-A., Zhang, S., Cruz-Munoz, M.-E., et al. (2017). SLAMF7 is critical for phagocytosis of haematopoietic tumour cells via Mac-1 integrin. Nature 544, 493-497.
Chow, C.-W., Downey, G. P., and Grinstein, S. (2004). Measurements of Phagocytosis and Phagosomal Maturation. Curr. Protoc. Cell Biol. 22, 15.7.1-15.7.33.
Dheilly, E., Moine, V., Broyer, L., Salgado-Pires, S., Johnson, Z., Papaioannou, A., Cons, L., Calloud, S., Majocchi, S., Nelson, R., et al. (2017). Selective Blockade of the Ubiquitous Checkpoint Receptor CD47 Is Enabled by Dual-Targeting Bispecific Antibodies. Mol. Ther. 25, 523-533.
Dransfield, I., Cabanas, C., Craig, A., and Hogg, N. (1992). Divalent Cation Regulation of the Function of the Leukocyte Integrin LFA1.
Dupuy, A. G., and Caron, E. (2008). Integrin-dependent phagocytosis: spreading from microadhesion to new concepts. J. Cell Sci. 121, 1773-1783.
Edelstein, A., Amodaj, N., Hoover, K., Vale, R., and Stuurman, N. (2010). Computer control of microscopes using manager. Curr. Protoc. Mol. Biol. Chapter 14, Unit14 20.
Fadok, V. A., Voelker, D. R., Campbell, P. A., Cohen, J. J., Bratton, D. L., and Henson, P. M. (1992). Exposure of phosphatidylserine on the surface of apoptotic lymphocytes triggers specific recognition and removal by macrophages. J. Immunol. 148, 2207-2216.
Farlow, J., Seo, D., Broaders, K. E., Taylor, M. J., Gartner, Z. J., and Jun, Y. W. (2013). Formation of targeted monovalent quantum dots by steric exclusion. Nat. Methods.
Freeman, S. A., and Grinstein, S. (2014). Phagocytosis: Receptors, signal integration, and the cytoskeleton. Immunol. Rev. 262, 193-215.
Freeman, S. A., Goyette, J., Furuya, W., Woods, E. C., Bertozzi, C. R., Bergmeier, W., Hinz, B., Van Der Merwe, P. A., Das, R., and Grinstein, S. (2016). Integrins Form an Expanding Diffusional Barrier that Coordinates Phagocytosis. Cell 164, 128-140.
Fujioka, Y., Matozaki, T., Noguchi, T., Iwamatsu, A., Yamao, T., Takahashi, N., Tsuda, M., Takada, T., and Kasuga, M. (1996). A novel membrane glycoprotein, SHPS-1, that binds the SH2-domain-containing protein tyrosine phosphatase SHP-2 in response to mitogens and cell adhesion. Mol. Cell. Biol. 16, 6887-6899.
Gardai, S. J., McPhillips, K. A., Frasch, S. C., Janssen, W. J., Starefeldt, A., Murphy-Ullrich, J. E., Bratton, D. L., Oldenborg, P.-A. A., Michalak, M., and Henson, P. M. (2005). Cell-surface calreticulin initiates clearance of viable or apoptotic cells through trans-activation of LRP on the phagocyte. Cell 123, 321-334.
Gardner, B., Anstee, D. J., Mawby, W. J., Tanner, M. J., and von dem Borne, A. E. (1991). The abundance and organization of polypeptides associated with antigens of the Rh blood group system. Transfus. Med. 1, 77-85.
Geiger, B., Spatz, J. P., and Bershadsky, A. D. (2009). Environmental sensing through focal adhesions. Nat. Rev. Mol. Cell Biol. 10, 21-33.
Gholamin, S., Mitra, S. S., Feroze, A. H., Liu, J., Kahn, S. A., Zhang, M., Esparza, R., Richard, C., Ramaswamy, V., Remke, M., et al. (2017). Disrupting the CD47-SIRPα anti-phagocytic axis by a humanized anti-CD47 antibody is an efficacious treatment for malignant pediatric brain tumors. Sci. Transl. Med. 9, eaaf2968.
Goodridge, H. S., Reyes, C. N., Becker, C. A., Katsumoto, T. R., Ma, J., Wolf, A. J., Bose, N., Chan, A. S. H., Magee, A. S., Danielson, M. E., et al. (2011). Activation of the innate immune receptor Dectin-1 upon formation of a ‘phagocytic synapse.’ Nature 472, 471-475.
Hatherley, D., Graham, S. C., Turner, J., Harlos, K., Stuart, D I., and Barclay, A. N. (2008). Paired Receptor Specificity Explained by Structures of Signal Regulatory Proteins Alone and Complexed with CD47. Mol. Cell 31, 266-277.
Hui, E., and Vale, R. D. (2014). In vitro membrane reconstitution of the T-cell receptor proximal signaling network. Nat. Struct. Mol. Biol. 21, 133-142.
Inagaki, K., Yamao, T., Noguchi, T., Matozaki, T., Fukunaga, K., Takada, T., Hosooka, T., Akira, S., and Kasuga, M. (2000). SHPS-1 regulates integrin-mediated cytoskeletal reorganization and cell motility. EMBO J. 19, 6721-6731.
Jacobs, S. A., Diem, M. D., Luo, J., Teplyakov, A., Obmolova, G., Malia, T., Gilliland, G. L., and O'Neil, K. T. (2012). Design of novel FN3 domains with high stability by a consensus sequence approach. Protein Eng. Des. Sel. 25, 107-117.
Jaiswal, S., Jamieson, C. H. M., Pang, W. W., Park, C. Y., Chao, M. P., Majeti, R., Traver, D., van Rooijen, N., and Weissman, I. L. (2009). CD47 Is Upregulated on Circulating Hematopoietic Stem Cells and Leukemia Cells to Avoid Phagocytosis. Cell 138, 271-285.
James, J. R., and Vale, R. D. (2012). Biophysical mechanism of T-cell receptor triggering in a reconstituted system. Nature 487, 64-69.
Jiang, P., Lagenaur, C. F., and Narayanan, V. (1999). Integrin-associated protein is a ligand for the P84 neural adhesion molecule. J. Biol. Chem. 274, 559-562.
Jones, S. L., Knaus, U. G., Bokoch, G. M., and Brown, E. J. (1998). Two signaling mechanisms for activation of alphaM beta2 avidity in polymorphonuclear neutrophils. J. Biol. Chem. 273, 10556-10566.
Kharitonenkov, A., Chen, Z., Sures, I., Wang, H., Schilling, J., and Ullrich, A. (1997). A family of proteins that inhibit signalling through tyrosine kinase receptors. Nature 386, 181-186.
Kojima, Y., Volkmer, J.-P., McKenna, K., Civelek, M., Lusis, A. J., Miller, C. L., Direnzo, D., Nanda, V., Ye, J., Connolly, A. J., et al. (2016). CD47-blocking antibodies restore phagocytosis and prevent atherosclerosis. Nature 536, 86-90.
Lehrman, E. K., Wilton, D. K., Litvina, E. Y., Welsh, C. A., Chang, S. T., Frouin, A., Walker, A. J., Heller, M. D., Umemori, H., Chen, C., et al. (2018). CD47 Protects Synapses from Excess Microglia-Mediated Pruning during Development. Neuron 100, 120-134.e6.
Lin, J., Kurilova, S., Scott, B. L., Bosworth, E., Iverson, B. E., Bailey, E. M., and Hoppe, A. D. (2016). TIRF imaging of Fc gamma receptor microclusters dynamics and signaling on macrophages during frustrated phagocytosis. BMC Immunol. 17, 5
Liu, D. Q., Li, L. M., Guo, Y. L., Bai, R., Wang, C., Bian, Z., Zhang, C. Y., and Zen, K. (2008). Signal regulatory protein a negatively regulates β 2 integrin-mediated monocyte adhesion, transendothelial migration and phagocytosis. PLoS One 3.
Liu, X., Pu, Y., Cron, K., Deng, L., Kline, J., Frazier, W. A., Xu, H., Peng, H., Fu, Y.-X., and Xu, M. M. (2015). CD47 blockade triggers T cell-mediated destruction of immunogenic tumors. Nat. Med. 21, 1209-1215.
Lu, J., Ellsworth, J. L., Hamacher, N., Oak, S. W., and Sun, P. D. (2011). Crystal Structure of Fey Receptor I and Its Implication in High Affinity γ-Immunoglobulin Binding. J. Biol. Chem. 286, 40608-40613.
Majeti, R., Chao, M. P., Alizadeh, A. A., Pang, W. W., Jaiswal, S., Gibbs, K. D., van Rooijen, N., and Weissman, I. L. (2009). CD47 Is an Adverse Prognostic Factor and Therapeutic Antibody Target on Human Acute Myeloid Leukemia Stem Cells. Cell 138, 286-299.
Matlung, H. L., Babes, L., Zhao, X. W., van Houdt, M., Treffers, L. W., van Rees, D. J., Franke, K., Schornagel, K., Verkuijlen, P., Janssen, H., et al. (2018). Neutrophils Kill Antibody-Opsonized Cancer Cells by Trogoptosis. Cell Rep. 23, 3946-3959.e6.
McCall, M. N., Shotton, D. M., and Barclay, A. N. (1992). Expression of soluble isoforms of rat CD45. Analysis by electron microscopy and use in epitope mapping of anti-CD45R monoclonal antibodies. Immunology 76, 310-317.
Michaels, A. D., Newhook, T. E., Adair, S. J., Morioka, S., Goudreau, B. J., Nagdas, S., Mullen, M. G., Persily, J. B., Bullock, T. N. J., Slingluffjr, C. L., et al. (2017). Cancer Therapy: Preclinical CD47 Blockade as an Adjuvant Immunotherapy for Resectable Pancreatic Cancer.
Motegi, S. -i., Okazawa, H., Ohnishi, H., Sato, R., Kaneko, Y., Kobayashi, H., Tomizawa, K., Ito, T., Honma, N., Buhring, H., et al. (2003). Role of the CD47-SHPS-1 system in regulation of cell migration. EMBO J. 22, 2634-2644.
Mouro-Chanteloup, I., Delaunay, J., Gane, P., Nicolas, V., Johansen, M., Brown, E. J., Peters, L. L., Van Kim, C. Le, Cartron, J. P., and Colin, Y. (2003). Evidence that the red cell skeleton protein 4.2 interacts with the Rh membrane complex member CD47. Blood 101, 338-344.
Noguchi, T., Matozaki, T., Fujioka, Y., Yamao, T., Tsuda, M., Takada, T., and Kasuga, M. (1996). Characterization of a 115-kDa protein that binds to SH-PTP2, a protein-tyrosine phosphatase with Src homology 2 domains, in Chinese hamster ovary cells. J. Biol. Chem. 271, 27652-27658.
O'Donoghue, G. P., Pielak, R. M., Smoligovets, A. A., Lin, J. J., and Groves, J. T. (2013). Direct single molecule measurement of TCR triggering by agonist pMHC in living primary T cells. Elife 2.
Okazawa, H., Motegi, S. -i., Ohyama, N., Ohnishi, H., Tomizawa, T., Kaneko, Y., Oldenborg, P.-A., Ishikawa, O., and Matozaki, T. (2005). Negative Regulation of Phagocytosis in Macrophages by the CD47-SHPS-1 System. J. Immunol. 174, 2004-2011.
Oldenborg, P.-A., Gresham, H. D., and Lindberg, F. P. (2001). Cd47-Signal Regulatory Protein a (Sirpα) Regulates Fcγ and Complement Receptor-Mediated Phagocytosis. J. Exp. Med. 193, 855-862.
Oldenborg, P. A., Zheleznyak, A., Fang, Y. F., Lagenaur, C. F., Gresham, H. D., and Lindberg, F. P. (2000). Role of CD47 as a marker of self on red blood cells. Science 288, 2051-2054.
Panni, R. Z., Herndon, J. M., Zuo, C., Hegde, S., Hogg, G. D., Knolhoff, B. L., Breden, M. A., Li, X., Krisnawan, V. E., Khan, S. Q., et al. (2019). Agonism of CD11b reprograms innate immunity to sensitize pancreatic cancer to immunotherapies. Sci. Transl. Med. 11, eaau9240.
Poon, I. K. H., Lucas, C. D., Rossi, A. G., and Ravichandran, K. S. (2014). Apoptotic cell clearance: basic biology and therapeutic potential. Nat. Rev. Immunol. 14, 166-180.
Rueden, C. T., Schindelin, J., Hiner, M. C., DeZonia, B. E., Walter, A. E., Arena, E. T., and Eliceiri, K. W. (2017). ImageJ2: ImageJ for the next generation of scientific image data. BMC Bioinformatics 18, 529.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: An open-source platform for biological-image analysis. Nat. Methods 9, 676-682.
Schmid, E. M., Bakalar, M. H., Choudhuri, K., Weichsel, J., Ann, H. S., Geissler, P. L., Dustin, M. L., and Fletcher, D. A. (2016). Size-dependent protein segregation at membrane interfaces. Nat. Phys. 12, 704-711.
Schmid, M. C., Khan, S. Q., Kaneda, M. M., Pathria, P., Shepard, R., Louis, T. L., Anand, S., Woo, G., Leem, C., Faridi, M. H., et al. (2018). Integrin CD11b activation drives anti-tumor innate immunity. Nat. Commun. 9, 5379.
Seiffert, M., Cant, C., Chen, Z., Rappold, I., Brugger, W., Kanz, L., Brown, E. J., Ullrich, A., and Bühring, H. J. (1999). Human signal-regulatory protein is expressed on normal, but not on subsets of leukemic myeloid cells and mediates cellular adhesion involving its counterreceptor CD47. Blood 94, 3633-3643.
Spencer, D. M., Wandless, T. J., Schreiber, S. L., and Crabtree, G. R. (1993). Controlling signal transduction with synthetic ligands. Science 262, 1019-1024.
Springer, T. A., and Dustin, M. L. (2011). Integrin inside-out signaling and the immunological synapse. Curr. Opin. Cell Biol. 24, 107-115.
Subramanian, S., Parthasarathy, R., Sen, S., Boder, E. T., and Discher, D. E. (2006). Species- and cell type-specific interactions between CD47 and human SIRP. Blood 107, 2548-2556.
Swaminathan, V., Kalappurakkal, J. M., Mehta, S. B., Nordenfelt, P., Moore, T. I., Koga, N., Baker, D. A., Oldenbourg, R., Tani, T., Mayor, S., et al. (2017). Actin retrograde flow actively aligns and orients ligand-engaged integrins in focal adhesions. Proc. Natl. Acad. Sci. 114, 10648-10653.
Tamada, M., Sheetz, M. P., and Sawada, Y. (2004). Activation of a Signaling Cascade by Cytoskeleton Stretch. Dev. Cell 7, 709-718.
Tsai, R. K., and Discher, D. E. (2008). Inhibition of “self” engulfment through deactivation of myosin-II at the phagocytic synapse between human cells. J. Cell Biol. 180, 989-1003.
Tseng, D., Volkmer, J.-P. J.-P., Willingham, S. B., Contreras-Trujillo, H., Fathman, J. W., Fernhoff, N. B., Seita, J., Inlay, M. A., Weiskopf, K., Miyanishi, M., et al. (2013). Anti-CD47 antibody-mediated phagocytosis of cancer by macrophages primes an effective antitumor T-cell response. Proc. Natl. Acad. Sci. 110, 11103-11108.
Tsuda, M., Matozaki, T., Fukunaga, K., Fujioka, Y., Imamoto, A., Noguchi, T., Takada, T., Yamao, T., Takeda, H., Ochi, F., et al. (1998). Integrin-mediated tyrosine phosphorylation of SHPS-1 and its association with SHP-2. Roles of Fak and Src family kinases. J. Biol. Chem. 273, 13223-13229.
Utsugi, T., Schroit, A. J., Connor, J., Bucana, C. D., and Fidler, I. J. (1991). Elevated expression of phosphatidylserine in the outer membrane leaflet of human tumor cells and recognition by activated human blood monocytes. Cancer Res. 51, 3062-3066.
Veillette, A., Thibaudeaut, E., and Latour, S. (1998). High expression of inhibitory receptor SHPS-1 and its association with protein-tyrosine phosphatase SHP-1 in macrophages. J. Biol. Chem. 273, 22719-22728.
Weischenfeldt, J., and Porse, B. (2008). Bone Marrow-Derived Macrophages (BMM): Isolation and Applications. CSH Protoc. 2008, pdb.prot5080.
Willingham, S. B., Volkmer, J.-P., Gentles, A. J., Sahoo, D., Dalerba, P., Mitra, S. S., Wang, J., Contreras-Trujillo, H., Martin, R., Cohen, J. D., et al. (2012). The CD47-signal regulatory protein alpha (SIRPα) interaction is a therapeutic target for human solid tumors. Proc. Natl. Acad. Sci. 109, 6662-6667.
Wong, H. S., Jaumouillé, V., Freeman, S. A., Doodnauth, S. A., Schlam, D., Canton, J., Mukovozov, I. M., Saric, A., Grinstein, S., and Robinson, L. A. (2016). Chemokine Signaling Enhances CD36 Responsiveness toward Oxidized Low-Density Lipoproteins and Accelerates Foam Cell Formation. Cell Rep. 14, 2859-2871.
Woollett, G. R., Williams, A. F., and Shotton, D. M. (1985). Visualisation by low-angle shadowing of the leucocyte-common antigen. A major cell surface glycoprotein of lymphocytes. EMBO J. 4, 2827-2830.
Wu, J., Wu, H., An, J., Ballantyne, C. M., and Cyster, J. G. (2018). Critical role of integrin CD11c in splenic dendritic cell capture of missing-self CD47 cells to induce adaptive immunity. Proc. Natl. Acad. Sci. 201805542.
Yi, T., Li, J., Chen, H., Hu, Y., Lowell, C. A., and Cyster Correspondence, J. G. (2015). Splenic Dendritic Cells Survey Red Blood Cells for Missing Self-CD47 to Trigger Adaptive Immune Responses. Immunity 43, 764-775.
Yu, D. H., Qu, C. K., Henegariu, O., Lu, X., and Feng, G. S. (1998). Protein-tyrosine phosphatase Shp-2 regulates cell spreading, migration, and focal adhesion. J. Biol. Chem. 273, 21125-21131.

What is claimed is:

1. A method for treating a cancer in an individual in need thereof, comprising administering to the individual:
(a) a first therapy comprising therapeutically effective amount of an integrin agonist; and
(b) a second therapy comprising a cancer therapy that targets at least one cancer-associated antigen and/or cancer-specific antigen.

2. The method of claim 1, wherein the integrin agonist activates one or more integrins selected from the group consisting of α integrins, β integrins, and combinations of any thereof.

3. The method of any one of claims 1 to 2, wherein the integrin agonist activates αVβ3, αLβ2, αMβ2, αXβ2, αDβ2, α4β1, α4β7, αEβ7, or a combination of any thereof.

4. The method of any one of claims 1 to 3, wherein the integrin agonist comprises a manganese treatment, a high affinity integrin ligand, a small molecule agonist, or a combination of any thereof.

5. The method of claim 4, wherein the small molecule agonist of integrin comprises leukadherin-1 (LA1), ADH-503, or a combination thereof.

6. The method of claim 4, wherein the high affinity integrin ligand comprises ICAM-1, ICAM-2, ICAM-3, VCAM-1, MAdCAM-1, E-cadherin, JAM-1, JAM-2, JAM-3, or a combination of any thereof.

7. The method of any one of claims 1 to 6, wherein the cancer is a solid tumor or a hematologic malignancy.

8. The method of claim 7, wherein the cancer is selected from the group consisting of leukemia, pancreatic cancer, a colon cancer, an ovarian cancer, a prostate cancer, a lung cancer, mesothelioma, a breast cancer, a urothelial cancer, a liver cancer, a head and neck cancer, a sarcoma, a cervical cancer, a stomach cancer, a gastric cancer, a melanoma, a uveal melanoma, a cholangiocarcinoma, multiple myeloma, lymphoma, and glioblastomas.

9. The method of any one of claims 1 to 8, wherein the targeted cancer therapy comprises an antibody therapy, a chimeric antigen receptor T-cell (CAR-T) therapy, a chimeric antigen receptor for phagocytosis (CAR-P) therapy, a myeloid-targeting therapy, or a combination thereof, which targets at least one cancer-associated antigen and/or cancer-specific antigen.

10. The method of claim 9, wherein the targeted cancer therapy comprises one or more phagocytic cells expressing a CAR that comprises an intracellular signaling domain of the engulfment receptor.

11. The method of claim 10, wherein the intracellular signaling domain of the engulfment receptor comprises at least 1, at least 2, at least 3, at least 4, or at least 5 ITAM motifs.

12. The chimeric polypeptide of any one of claims 9 to 10, wherein the intracellular signaling domain from the engulfment receptor is capable of mediating endogenous phagocytic signaling pathway.

13. The chimeric polypeptide of any one of claims 9 to 12, the engulfment receptor is selected from the group consisting of Megf10, FcRγ, Bai1, MerTK, TIM4, Stabilin-1, Stabilin-2, RAGE, CD300f, Integrin subunit αv, Integrin subunit 05, CD36, LRP1, SCARF1, C1Qa, and Ax1.

14. The method of any one of claims 10 to 13, wherein the one or more phagocytic cells is selected from the group consisting of macrophages, dendritic cells, mast cells, monocytes, neutrophils, microglia, and astrocytes.

15. The method of claim 14, wherein at least one of the one or more phagocytic cells is a bone marrow derived macrophage (BMDM) or a bone marrow derived dendritic cell (BMDC).

16. The method of claim 9, wherein the targeted cancer therapy is an antibody therapy comprising an anti-CD47 antibody, an anti-SIRPα antibody, or a combination thereof.

17. The method of any one of claims 1 to 16, wherein the first therapy and the second therapy are administered concomitantly.

18. The method of any one of claims 1 to 16, wherein the first therapy is administered at the same time as the second therapy.

19. The method of any one of claims 1 to 16, wherein the first therapy and the second therapy are administered sequentially.

20. The method of claim 19, wherein the first therapy is administered before the second therapy.

21. The method of claim 19, wherein the first therapy is administered after the second therapy.

22. The method of claim 17, wherein the first therapy is administered before and/or after the second therapy.

23. The method of claim 17, wherein the first therapy and the second therapy are administered in rotation.

24. The method of claim 17, wherein the first therapy and the second therapy are administered together in a single formulation.

25. A kit for treating a cancer in a subject in need thereof, the kit comprising one or more integrin agonists and instructions for use of the one or more integrin agonists in combination with a cancer therapy that targets a cancer-associated antigen and/or a cancer-specific antigen.
